(19) United States
(12) Patent Application Publication
(10) Pub. No.: US 2025/0235478 A1
(43) Pub. Date: Jul. 24, 2025

(54) CHIMERIC ANTIGEN RECEPTOR MODIFIED REGULATORY T CELLS FOR TREATING CANCER

(71) Applicant: MUSC FOUNDATION FOR RESEARCH DEVELOPMENT, Charleston, SC (US)

(72) Inventor: Leonardo M.R. FERREIRA, Charleston, SC (US)

(73) Assignee: MUSC FOUNDATION FOR RESEARCH DEVELOPMENT, Carleston, SC (US)

(21) Appl. No.: 18/853,555

(22) PCT Filed: Apr. 25, 2023
(86) PCT No.: PCT/US2023/019852

§ 371 Date: Oct. 2, 2024

Related U.S. Application Data
(60) Provisional application No. 63/336,137, filed on Apr. 28, 2022.

(51) Int. Cl. A61K 35/17; A61K 40/11; A61K 40/31; A61K 40/42; A61K 45/06; A61P 35/00; C07K 14/52; C07K 14/525; C07K 14/535; C07K 14/54; C07K 14/55; C07K 14/57; C07K 14/705; C07K 14/725; C12N 5/0783; C12N 9/64; C12N 15/86

(52) U.S. Cl. CPC A61K 35/17; A61K 40/11; A61K 40/31; A61K 40/4211; A61K 45/06; A61P 35/00; C07K 14/521; C07K 14/525; C07K 14/535; C07K 14/5403; C07K 14/5412; C07K 14/5425; C07K 14/5428; C07K 14/5443; C07K 14/55; C07K 14/57; C07K 14/7051; C07K 14/70521; C12N 5/0637; C12N 9/6467; C12N 15/86; A61K 2239/13; A61K 2239/22; A61K 2239/38; C12N 2740/15043

(57) ABSTRACT

The present disclosure provides CAR regulatory T cells. Further provided herein are methods for treating cancer, such as a solid cancer, comprising administering an effective amount of CAR regulatory T cells.

### PRIORITY CLAIM

This application claims benefit of priority to U.S. Provisional Application Ser. No. 63/336,137, filed Apr. 28, 2022, the entire contents of which are hereby incorporated by reference.

### BACKGROUND

### 1. Field

The disclosure relates generally to the field of molecular biology. More particularly, it concerns regulatory T cells with a chimeric antigen receptor and methods of use thereof.

### 2. Related Art

The adaptive immune system has evolved to specifically recognize and destroy a virtually infinite variety of pathogens, while remaining unresponsive towards self-tissues, a state known as immune tolerance. Regulatory T cells (T_{regs}) play a pivotal role in inducing and maintaining tolerance (1). Manipulating human Tr_{egs} has the potential to restore tolerance to treat autoimmunity and organ transplant rejection. Preclinical studies have shown that antigen-specific T_{regs} can reverse autoimmune diabetes in the mouse (6). Yet, vanishingly low abundance of antigen-specific T_{regs} and T_{reg} instability upon prolonged expansion have hampered the implementation of T_{reg}-based adoptive cell therapies. Moreover, the antigens recognized by T_{regs} remain largely unknown, impeding progress in the field.

Chimeric antigen receptor (CAR) technology has greatly expedited the generation of antigen-specific T cells for cancer therapy. CARs are synthetic receptors comprising an extracellular antigen-binding domain and an intracellular signaling domain, enabling T cell activation by an antigen of choice. CAR T cell therapies, FDA-approved to treat B cell malignancies, have led to remission rates higher than with any previously approved drug, transforming cancer treatment (7). A CAR can be designed to redirect a Treg to a specific target antigen. For instance, for type 1 diabetes, an autoimmune disorder where the insulin-producing beta cells of the pancreas are destroyed by autoreactive T cells, one could use a CAR to target Tregs directly to inflamed islets.

Importantly, T_{regs} are also an emerging target in cancer immunotherapy. Thymically derived T_{regs} migrate from the peripheral blood to and accumulate in the tumor microenvironment, constituting one of the barriers to cancer immunotherapy (2,5). Of note, cytotoxicity has been found to be one of the multiple mechanisms utilized by T_{regs} to suppress immune responses. For instance, both granzyme B and perforin have been found to be required for optimal T_{reg} suppression of tumor clearance by either eliminating antigen presenting cells (APCs) or CD8₊T cells and NK cells directly (8-10).

As conventional T cells either fail to penetrate solid tumor or fail to function once in the tumor microenvironment, whereas T_{regs} migrate to and prosper in solid tumors (2,5), generating CAR T_{regs} that recognize solid tumor cells directly can greatly improve engineered immune cell therapies for cancer. Thus, there is an unmet need for determining the mechanisms behind the inflammatory and cytotoxic properties of tumor-targeting CAR T_{regs}.

### SUMMARY

In certain embodiments, the present disclosure provides methods treating cancer in a subject comprising administering to the subject an effective amount of chimeric antigen receptor (CAR) regulatory T cells (T_{regs}).

In some aspects, the CAR comprises a CD28-CD3ξ intracellular domain. In certain aspects, the CAR binds a tumor-associated antigen. For example, the tumor-associated antigen may be, but is not limited to, CD19, CD20, CD22, B-cell maturation antigen (BCMA), carcinoembryonic antigen (CEA), alphafetoprotein, CA-125, MUC-1, epithelial tumor antigen, melanoma-associated antigen (MAGE), mutated p53-derived peptide-HLA, mutated ras-derived peptide-HLA, HER2/Neu, ERBB2, folate binding protein, HIV-1 envelope glycoprotein gp120, HIV-1 envelope glycoprotein gp41, GD2, CD123 (IL3RA), CD319, CD23, CD30, CD56, c-Met, mesothelin, GD3, HERV-K, IL-11Ralpha, kappa chain, lambda chain, CSPG4, ERBB2, EGFR, EGFRvIII, VEGFR2, TNFRSF17, SDC1, FAP, CD44, MS4A1, EPCAM, CA9, CD174, TNFRSF8, CD33, CD38, EPHA2, CD248, CD274, CD276, CD5, NCAM1, CD70, ERBB2, KDR, L1CAM, ULBP1, ULBP2, IL1RAP, GPC3, IL13RA2, ROR1, CEACAM5, MET, FOLH1, CSPG4, CD133, GPNMB, or PSCA.

In certain aspects, the T_{regs} are human T_{regs}. In some aspects, the human T_{regs} were isolated from human peripheral blood by sorting for CD4⁺CD25^{high}CD127^{low} cells.

In some aspects, the cancer is acute lymphoblastic leukemia (ALL), B cell leukemia, myeloid leukemia, or epithelial lung carcinoma. In certain aspects, the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

In some aspects, the T cells and/or at least one additional therapeutic agent is administered intravenously, intrapcritoncally, intratrachcally, intratumorally, intramuscularly, endoscopically, intralesionally, percutaneously, subcutaneously, regionally, or by direct injection or perfusion. In specific aspects, the CAR T_{regs} are delivered intravenously or subcutaneously.

In certain aspects, the CAR T_{regs} express IFN-γ, TNF-α, perforin and/or granzyme B. In some aspects, the CAR T_{regs} express pro-inflammatory cytokines IFN-γ, IL-3, CXCL9, CXCL11, IL-2, IL-9, IL-17A, CSF3, CCL3, TNFα, and/or IL-6. In some aspects, the CAR T_{regs} express cytolytic proteins granzyme A, granzyme B, perforin 1 (PRF1), NKG7, and/or granzyme H. In particular aspects, the CAR T_{regs} secrete IL-10. In specific aspects, the CAR T_{regs} express FOXP3, CD25, BATF, ICOS, GITR, and/or a demethylated T_{reg}-specific demethylated region (TSDR).

In further aspects, the CAR T_{regs} are conjugated to a cytotoxic agent. In some aspects, the cytotoxic agent is a chemotherapeutic, IL-2, IL-15, soluble TRAIL, perforin, or granzyme B.

In some aspects, the method further comprises administering at least a second anticancer therapy to the subject. In certain aspects, the second anticancer therapy is a surgical therapy, chemotherapy, radiation therapy, cryotherapy, hormonal therapy, immunotherapy or cytokine therapy. In some aspects, the CAR T_{regs} are administered more than once.

A further embodiment provides a composition comprising T_{regs} engineered to express a CAR construct. In some aspects, the CAR construct comprises a tumor-associated antigen antibody or fragment thereof selected from the group consisting of F(ab′)2, Fab′, Fab, Fv, and scFv. In certain aspects, the CAR binds a tumor-associated antigen. For example, the tumor-associated antigen may be, but is not limited to, CD19, CD20, CD22, B-cell maturation antigen (BCMA), carcinoembryonic antigen (CEA), alphafetoprotein, CA-125, MUC-1, epithelial tumor antigen, melanoma-associated antigen (MAGE), mutated p53-derived peptide-HLA, mutated ras-derived peptide-HLA, HER2/Neu, ERBB2, folate binding protein, HIV-1 envelope glycoprotein gp120, HIV-1 envelope glycoprotein gp41, GD2, CD123 (IL3RA), CD319, CD23, CD30, CD56, c-Met, mesothelin, GD3, HERV-K, IL-11Ralpha, kappa chain, lambda chain, CSPG4, ERBB2, EGFR, EGFRvIII, VEGFR2, TNFRSF17, SDC1, FAP, CD44, MS4A1, EPCAM, CA9, CD174, TNFRSF8, CD33, CD38, EPHA2, CD248, CD274, CD276, CD5, NCAM1, CD70, ERBB2, KDR, L1CAM, ULBP1, ULBP2, IL1RAP, GPC3, IL13RA2, ROR1, CEACAM5, MET, FOLH1, CSPG4, CD133, GPNMB, or PSCA. In certain aspects, the T_{regs} are autologous. In some aspects, the T_{regs} are allogeneic. In some aspects, the T_{regs} express pro-inflammatory cytokines IFN-γ, IL-3, CXCL9, CXCL11, IL-2, IL-9, IL-17A, CSF3, CCL3, TNFα, and/or IL-6. In particular aspects, the T_{regs} express cytolytic proteins granzyme A, granzyme B, perforin 1 (PRF1), NKG7, and/or granzyme H. In some aspects, the T_{regs} secrete IL-10. In some aspects, the T_{regs} express FOXP3, CD25, BATF, ICOS, GITR, and/or a demethylated T_{reg}-specific demethylated region (TSDR). In specific aspects, the composition is essentially free of CD8⁺ T cells. In further aspects, the T_{regs} are conjugated to a cytotoxic agent. In some aspects, the cytotoxic agent is a chemotherapeutic.

In yet another embodiment, there is provided a pharmaceutical composition comprising the T_{regs} of the present embodiments and aspects thereof and a pharmaceutical carrier. Also provided herein is a composition comprising an effective amount of T_{regs} of any of the present embodiments and aspects thereof for use in the treatment of cancer in a subject.

Another embodiment provides an in vitro method of generating CAR T_{regs} comprising (a) isolating T_{regs} from peripheral blood; (b) introducing a CAR expression construct to the Tregs; (c) expanding the T_{regs} in the presence of at least one cytokine; and (d) stimulating the T_{regs} with artificial presenting cells (APCs).

In some aspects, the CAR expression construct is a lentiviral vector or retroviral vector. In certain aspects, introducing comprises contacting the T_{regs} with lentiviral particles comprising a CAR construct. In certain aspects, the at least one cytokine is IL-2. In some aspects, the APCs are gamma-irradiated APCs. In certain aspects, the APCs are CD19-K562 cells. In some aspects, the CAR expression construct is a CD19-specific construct. For example, the CAR may be, but is not limited to, a CD19, CD20, CD22, B-cell maturation antigen (BCMA), carcinoembryonic antigen (CEA), alphafetoprotein, CA-125, MUC-1, epithelial tumor antigen, melanoma-associated antigen (MAGE), mutated p53-derived peptide-HLA, mutated ras-derived peptide-HLA, HER2/Neu, ERBB2, folate binding protein, HIV-1 envelope glycoprotein gp120, HIV-1 envelope glycoprotein gp41, GD2, CD123 (IL3RA), CD319, CD23, CD30, CD56, c-Met, mesothelin, GD3, HERV-K, IL-11Ralpha, kappa chain, lambda chain, CSPG4, ERBB2, EGFR, EGFRvIII, VEGFR2, TNFRSF17, SDC1, FAP, CD44, MS4A1, EPCAM, CA9, CD174, TNFRSF8, CD33, CD38, EPHA2, CD248, CD274, CD276, CD5, NCAM1, CD70, ERBB2, KDR, L1CAM, ULBP1, ULBP2, IL1RAP, GPC3, IL13RA2, ROR1, CEACAM5, MET, FOLH1, CSPG4, CD133, GPNMB, or PSCA construct.

It is contemplated that any method or composition described herein can be implemented with respect to any other method or composition described herein. For example, a compound synthesized by one method may be used in the preparation of a final compound according to a different method.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Mechanisms of immune suppression by regulatory T cells (Tregs). Tregs carry their immunosuppressive function using different mechanisms, including: 1) Tregs secrete the anti-inflammatory cytokine IL-10, inducing a tolerogenic state in antigen-presenting cells (APCs). Tolerogenic APCs in turn interact with CD4+ T effector (Teff) cells and inhibiting their secretion of pro-inflammatory cytokines. In addition, tolerogenic APCs induce the differentiation of naïve T cells into Tregs. 2) IL-2 secreted by Teff cells and essential for their activation and proliferation is taken up by Treg cells via CD25. Tregs also induce PD-L1 expression in APCs, which then induce apoptosis in activated PD-1+ Teff cells via PD-1/PD-L1 signaling. 3) Tregs induce apoptosis of APCs, such as B cells and dendritic cells (DCs), via perforin/granzyme-mediated cytotoxicity. 4) Tregs convert extracellular ATP into adenosine (ADO), a potent immunosuppressant, using ectoenzymes CD39 and CD73. ADO binds to its receptor in Teff cells, inhibiting them.

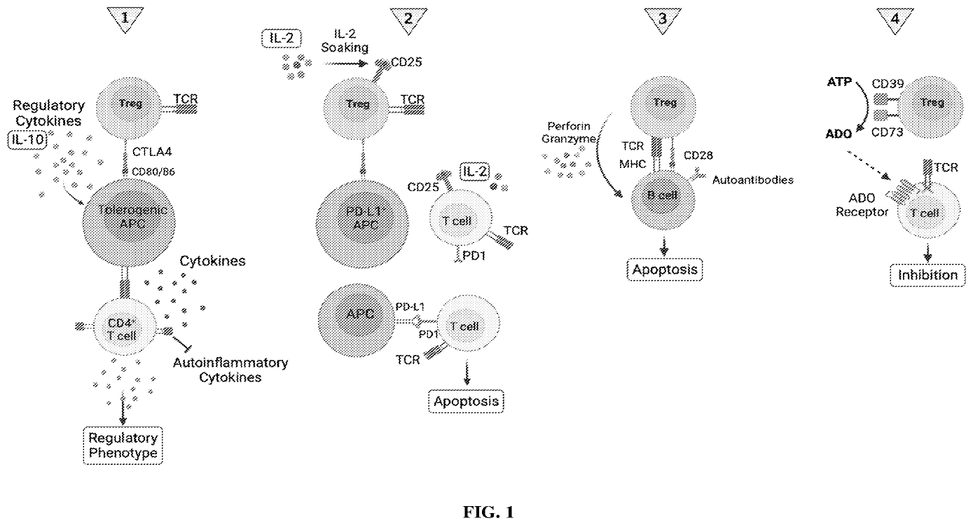

FIG. 2: T cell activation via T cell receptor (TCR) and CD28 vs. via chimeric antigen receptor (CAR). T cells require two signals to be fully activated. Signal 1 is provided by engagement of the T cell receptor (TCR) with its cognate antigen presented by a major histocompatibility complex (MHC) molecule, also known as human leukocyte antigen (HLA) in humans on the surface of the target cells, e.g., APCs. Of note, the TCR does not itself have signaling motifs, but it associates with CD3 molecules, namely CD3delta, CD3epsilon, CD3gamma, and CD3zeta, whose intracellular domains get phosphorylated and initiate a signaling cascade. Signal 2, also known as co-stimulation, is provided by engagement of CD28 on the T cell with CD80 or CD86 on the APC. A chimeric antigen receptor (CAR) is an artificial receptor that combines an extracellular antigen binding domain, typically a single chain fragment variable (scFv) domain, and an intracellular signaling domain, typically a tandem CD28-CD3zeta. This way, a CAR combines antigen recognition, T cell signal 1, and T cell signal 2 in a single molecule.

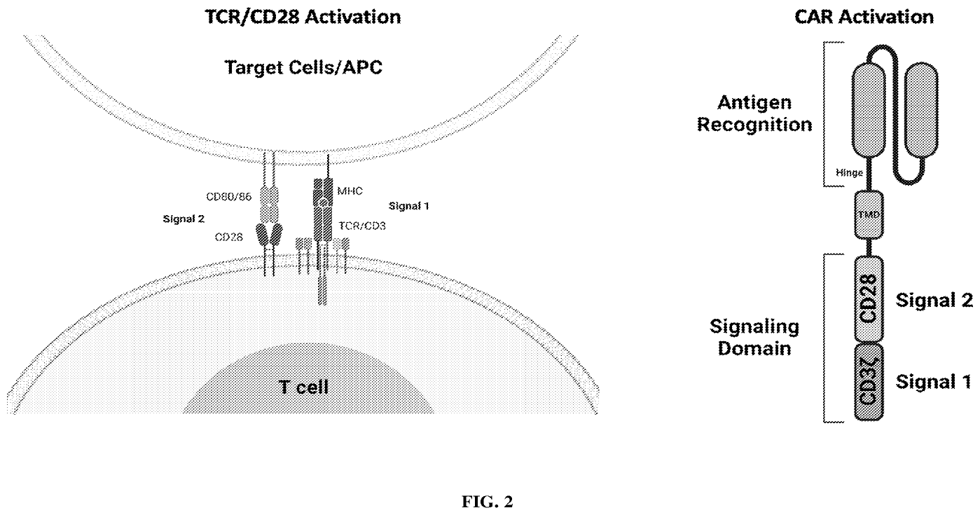

FIGS. 3A-3G: Chimeric antigen receptor signaling confers both regulatory and effector cell properties to human Tregs. (FIG. 3A) CD19+ NALM6 leukemia cell apoptosis induced by CD19 CAR Tregs in vitro. Annexin V labels apoptotic cells and DAPI labels dead cells. (FIG. 3B) CD19 CAR Treg single-cell protein expression of pro-inflammatory cytokines and cytolytic molecules in vitro. (FIG. 3C) CD19 CAR Tregs control luciferase-labeled NALM6 growth when co-injected in vivo in NSG mice. (FIG. 3D) CD19 CAR Tregs suppress CD19 CAR Teff cell proliferation in vitro. (FIG. 3E) CD19 CAR Tregs retain FOXP3 and HELIOS expression upon activation by CD19+ tumor cells. (FIG. 3F) CD19 CAR Tregs are cytotoxic towards GFP-labeled CD19-A549 lung cancer cells in vitro. Green, GFP (CD19-A549 cells); Blue, DAPI (nuclei), Violet, PI (dead cells). (FIG. 3G) CD19 CAR Tregs control luciferase-labeled CD19-A549 growth when co-injected in vivo in NSG mice.

FIG. 4: Dissecting CAR-induced gene expression programs in human Tregs using RNA sequencing. Human CD4+CD25+CD127-Tregs and CD4+CD25-CD127+ Teff cells were purified from human peripheral blood, activated, and transduced with CD19CAR-2A-GFP lentivirus. The modified cells were then sorted to purity, based on GFP expression, and co-incubated at a 1:1 ratio with irradiated K562 (No Activation), OKT3-loaded CD64-CD80-K562 (TCR/CD28 activation), or CD19-K562 (CAR activation). One day later, CD4+ cells were magnetically purified and used to generate bulk RNA sequencing libraries. Libraries were sequenced and the data analyzed using a Seurat pipeline. Of note, this CD19CAR construct features a CD28-CD3zeta signaling domain and OKT3 is an agonistic anti-CD3 antibody, while CD80 is a natural agonistic ligand for CD28. Therefore, T cell signal 1 (TCR/CD3) and T cell signal 2 (CD28) were compared being delivered via the CAR with being delivered via endogenous TCR/CD28.

FIG. 5: Global gene expression analysis of CAR and TCR/CD28-activated Treg and Teff cells using RNA sequencing. Left-hand side: Gene expression heatmap with all 6 conditions measured by RNA-seq (NoAct Treg, CAR Treg, TCR Treg, NoAct Treg, CAR Treg, TCR Treg) with 1000 most differentially expressed genes. Note that CAR Treg and CAR Teff cluster closer together (activation mode), whereas NoAct Treg and TCR Treg, as well as NoAct Teff and TCR Teff cluster together (cell type), indicating that CAR activation confers Teff cell gene programs to Tregs. Right-hand side: Gene expression heatmap with 200 most differentially expressed genes between CAR Tregs and TCR Tregs. Noteworthy genes upregulated in CAR Tregs in comparison to TCR Tregs include the cytokine/chemokine genes IL5, CCL4, CSF2, and the transcriptional regulator genes TBX21, IRF8, and ZBED2. NoAct, No Activation.

FIG. 6: Gene expression differences between CAR and TCR/CD28 activated Tregs. Expression levels of representative genes involved in Treg identity, suppressive function, and cytotoxicity, as well as inflammatory cytokine and chemokine genes. CAR Tregs have similar levels of Treg identity and suppressive function genes (with the exception of IL-10 and EBI3, a subunit of IL-35, which are higher in CAR Tregs) to TCR Tregs, yet have higher levels of cytotoxicity (GZMB, GZMH, NKG7) and inflammatory cytokine and chemokine genes (e.g. IFNG, IL3, TNF).

FIG. 7: CAR Tregs upregulate inflammatory genes and associated pathways. Left-hand side: Top 20 most upregulated genes in CAR Tregs vs. NoAct Tregs and in CAR Tregs vs. TCR Tregs in RNA-seq. Genes upregulated in both pairwise comparisons include IL3, CXCL11, and IFNG. Right-hand side: Gene set enrichment analysis (GSEA) of CAR vs. TCR/CD28 activation in Tregs. Top pathways upregulated in CAR Tregs included TNF, IL6, IFNG, and inflammation. FC, fold-change; pval, p-value; FDR, false discovery rate.

FIG. 8: CAR Teff cells upregulate inflammatory genes and associated pathways. Left-hand side: Top 20 most upregulated genes in CAR Teff vs. NoAct Teff and in CAR Teff vs. TCR Teff in RNA-seq. Genes upregulated in both pairwise comparisons include IL2 and CSF2, but not IFNG. In CAR Teff vs NoAct Teff, IFNG is one of the top 20 genes. Right-hand side: Gene set enrichment analysis (GSEA) of CAR vs. TCR/CD28 activation in Teff cells. Top pathways upregulated in CAR Teff cells included TNF, IL6, and inflammation, but not the IFNG pathway. Other pathways upregulated include apoptosis and the p53 pathway. FC, fold-change; pval, p-value; FDR, false discovery rate.

FIG. 9: Changes in gene expression with CAR and TCR/CD28 mediated activation of Tregs at the whole transcriptome level using RNA-seq. Volcano plots of gene expression for CAR Treg vs. NoAct Treg, TCR Treg vs. NoAct Treg, and CAR Treg vs. TCR Treg pairwise comparisons. The color code is as follows: red means upregulated, green means downregulated, and black means not statistically significant. FC, fold-change.

FIG. 10: Changes in gene expression with CAR and TCR/CD28 mediated activation of Teff cells at the whole transcriptome level using RNA-seq. Volcano plots of gene expression for CAR Teff vs. NoAct Teff, TCR Teff vs. NoAct Teff, and CAR Teff vs. TCR Teff pairwise comparisons. The color code is as follows: red means upregulated, green means downregulated, and black means not statistically significant. FC, fold-change.

FIG. 11: Dissecting CAR-induced gene expression programs in human Tregs using single-cell RNA sequencing. Human CD4+CD25+CD127-Tregs and CD4+CD25-CD127+Teff cells were purified from human peripheral blood, activated, and transduced with CD19CAR-2A-GFP lentivirus. The modified cells were then sorted to purity, based on GFP expression, and co-incubated at a 1:1 ratio with irradiated K562 (No Activation), OKT3-loaded CD64-CD80-K562 (TCR/CD28 activation), or CD19-K562 (CAR activation). One day later, CD4+ cells were magnetically purified and used to generate 10x Genomics single-cell RNA sequencing libraries. Libraries were sequenced and the data analyzed using a Seurat pipeline. Of note, this CD19CAR construct features a CD28-CD3zeta signaling domain and OKT3 is an agonistic anti-CD3antibody, while CD80 is a natural agonistic ligand for CD28. Therefore, T cell signal 1 (TCR/CD3) and T cell signal 2 (CD28) were compared being delivered via the CAR with being delivered via endogenous TCR/CD28.

FIG. 12: Single-cell RNA sequencing of activated CD4+ T cells. A total of 153,636 human CD4+ T cells from three independent donors were single-cell sequenced (79,386 Tregs and 74,250 Teff cells). Single-cell sequencing data was aligned and processed using a Seurat pipeline. Data was filtered for low mitochondrial gene content and adequate nUMI (number of unique molecular identifiers), selected for CD4+ T cells and clustered. A gene expression cluster arose that corresponded to contaminating K562 target cells. This cluster was excluded and the remaining cleaned data re-clustered. Left-hand: UMAP (Uniform Manifold Approximation and Projection) graphic with single cells colored by gene expression Seurat cluster at resolution 0.7 (14 clusters). Right-hand: UMAP graphic with single cells colored by condition—CAR Treg, TCR Treg, NoAct Treg, CAR Teff, TCR Teff, NoAct Teff (6 conditions). NoAct, no activation.

FIG. 13: Magnitude of activation of CD4+ T cells via CAR and via TCR/CD28 using single-cell RNA sequencing. An activation signature was computed by combining the top 20genes expressed in activated CD4+ T cells (CAR and TCR) in comparison to resting CD4+ T cells (NoAct). Left-hand: Violin plot of activation signature expression score for CAR CD4+ T cells, TCR CD4+ T cells, and NoAct CD4+ T cells. Right-hand: Top 20 upregulated genes in activated cells (Treg, Teff, total CD4+ T cells) and unactivated cells (Treg, Teff, CD4+ T cells).

FIG. 14: Magnitude of activation of single-cell gene expression CD4+ T cell clusters using single-cell RNA sequencing. An activation signature was computed by combining the top 20 genes expressed in activated CD4+ T cells (CAR and TCR) in comparison to resting CD4+ T cells (NoAct). Left-hand: Violin plot of activation signature expression score for single-cell clusters among total CD4+ T cells. Right-hand: cell proportion by condition (CAR Treg, TCR Treg, NoAct Treg, CAR Teff, TCR Teff, NoAct Teff) among CD4+ T cell single-cell gene expression clusters.

FIG. 15: Single-cell RNA sequencing of activated Tregs. Tregs were clustered based on single-cell gene expression. The leftmost panel is a UMAP plot colored by activation mode (NoAct, CAR, TCR). The middle panel is a UMAP plot colored by single-cell gene expression cluster (0-12). The rightmost panel is a stacked bar chart with the cell proportion by condition among single-cell gene expression clusters. Clusters 1, 2, 6, and 10 are enriched in NoAct Tregs. Clusters 7 and 9 are enriched in CAR Tregs. Clusters 3,4, and 11 are enriched in TCR Tregs.

FIG. 16: Magnitude of activation of Tregs via CAR and via TCR/CD28 using single-cell RNA sequencing. An activation signature was computed by combining the top 20 genes expressed in activated Tregs (CAR and TCR) in comparison to resting Tregs (NoAct). Left-hand: Violin plot of activation signature expression score for CAR Tregs, TCR Tregs, and NoAct Tregs. Right-hand: Top 20 upregulated genes in activated cells (Treg, Teff, total CD4+ T cells) and unactivated cells (Treg, Teff, CD4+ T cells).

FIG. 17: Magnitude of activation of single-cell gene expression Treg clusters using single-cell RNA sequencing. An activation signature was computed by combining the top 20 genes expressed in activated Tregs (CAR and TCR) in comparison to resting Tregs (NoAct). Left-hand: Violin plot of activation signature expression score for single-cell clusters among total Tregs. Right-hand: cell proportion by condition (CAR Treg, TCR Treg, NoAct Treg) among Treg single-cell gene expression clusters.

FIG. 18: Single-cell RNA sequencing of activated Teff cells. Teff cells were clustered based on single-cell gene expression. The leftmost panel is a UMAP plot colored by activation mode (NoAct, CAR, TCR). The middle panel is a UMAP plot colored by single-cell gene expression cluster (0-14). The rightmost panel is a stacked bar chart with the cell proportion by condition among single-cell gene expression clusters. Clusters 4, 5, 6, and 7 are enriched in NoAct Teff cells. Clusters 0,1, and 10 are enriched in CAR Teff cells. Clusters 2, 3, 8, and 9 are enriched in TCR Teff cells.

FIG. 19: Magnitude of activation of Teff cells via CAR and via TCR/CD28 using single-cell RNA sequencing. An activation signature was computed by combining the top 20genes expressed in activated Teff cells (CAR and TCR) in comparison to resting Teff cells (NoAct). Left-hand: Violin plot of activation signature expression score for CAR Teff, TCR Teff and NoAct Teff. Right-hand: Top 20 upregulated genes in activated cells (Treg, Teff, total CD4+ T cells) and unactivated cells (Treg, Teff, CD4+ T cells).

FIG. 20: Magnitude of activation of single-cell gene expression Teff cell clusters using single-cell RNA sequencing. An activation signature was computed by combining the top 20 genes expressed in activated Teff cells (CAR and TCR) in comparison to resting Teff cells (NoAct). Left-hand: Violin plot of activation signature expression score for single-cell clusters among total Teff cells. Right-hand: cell proportion by condition (CAR Treg, TCR Treg, NoAct Treg, CAR Teff, TCR Teff, NoAct Teff) among Teff cell single-cell gene expression clusters.

FIG. 21: Expression of cytotoxicity genes in Tregs activated via CAR or TCR/CD28 at the single-cell level. Levels of PRF1, GZMA, GZMB, FASLG, TNFRSF10A, TNF gene expression in UMAP plot of Tregs (NoAct, CAR, TCR).

FIG. 22: Expression of cytotoxicity genes in Tregs activated via CAR or TCR/CD28 at the single-cell gene expression cluster level. Cluster 6 (enriched in NoAct Treg) has highest GZMA and GZMK levels, cluster 9 (enriched in CAR Treg) has highest GMZB, FASLG, GZMH, and NKG7 levels, and cluster 12 (equal distribution of NoAct, CAR, and TCR Treg) has highest TNF, SERPINB9, PRF1, TNFRSF10A, and LAMP1 levels.

FIG. 23: CAR Treg cytotoxicity is perforin-dependent. Left-hand: PRF1 CRISPR KO CD19 CAR Tregs displayed reduced cytotoxicity towards NALM6 cells in vitro. Left-hand: PRF1CRISPR KO efficiency (59.3%) measured by the insertion/deletion frequency at the genomic level in CAR Tregs, assessed by TIDE (Tracking of Indels by Decomposition) analysis.

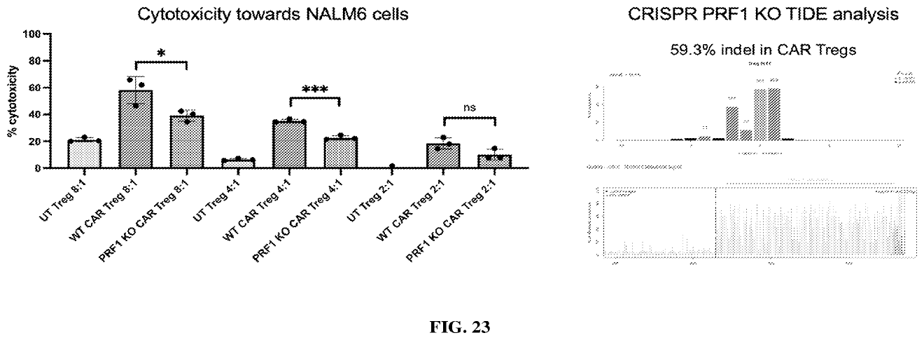

FIG. 24: Expression of cytokine and chemokine genes in Tregs activated via CAR or TCR/CD28 at the single-cell level. Levels of IFNG, IL5, CCL3, IL3, CSF2, CSF3, IL17A, and IL2 gene expression in UMAP plot of Tregs (NoAct, CAR, TCR). Note that expression of inflammatory cytokine and chemokine genes is mostly restricted to the CAR Treg region of the UMAP plot.

FIG. 25: Expression of cytokine and chemokine genes in Tregs activated via CAR or TCR/CD28 at the single-cell gene expression cluster level. Cluster 9 (enriched in CAR Treg) has highest levels of most inflammatory cytokine and chemokine genes.

FIG. 26: IFNG is expressed in both CAR and TCR Teff cells, but only in CAR Tregs at the single-cell level.

FIG. 27: Cytokine secretion by Tregs and Teff cells activated via CAR or TCR/CD28. The secretion of 48 different cytokines by Tregs and Teff (NoAct, CAR, TCR) was detected using multiplex ELISA. Cytokines higher in CAR Treg than TCR Treg and NoAct Treg (CAR exacerbated upregulation): sCD40L, FGF-2, Fractalkine, G-CSF, GM-CSF, GROa, IFN-a2, IFNg, IL-3, IL-4, IL-6, IL-9, IL-12p40, IL-12p70, IL-13, IL-17A, IL-18, MCP-1, MCP-3, MIG/CXCL9, MIP1a, PDGF-AA, TNFb. Cytokines higher in CAR Treg but not in TCR Treg than NoAct Treg (CAR specific upregulation): sCD40L, G-CSF, GM-CSF, GROa, IFN-a2, IFNg, IL-3, IL-4, IL-6, IL-12p40, IL-13, MIP1a. Cytokines lower in CAR Treg than TCR Treg: MIP1b, VEGF-A. Cytokines higher in CAR Teff than TCR Teff: IP-10, IL-17A, TNFb. Cytokines higher in CAR Teff but not in TCR Teff than NoAct Teff: None. Cytokines lower in CAR Teff but not in TCR Treg than NoAct Teff: None. Cytokines lower in CAR Teff than TCR Teff: sCD40L, GROa, IFNa2, IL-6, IL-10, IL-13, IL-15, MCP1, PDGF-AA, PDGF-AB/BB, VEGF-A. K562 signifies NoAct, 19K562 signifies CAR activation, and OKT3K562 signifies TCR/CD28 activation. *, p<0.05; **, p<0.01; ***, p<0.001; ****, p<0.0001.

FIG. 28: IFNG production by Tregs and Teff cells activated via CAR or TCR/CD28. Intracellular staining for the Treg transcription factor FOXP3 and cytokines in cells activated overnight and then treated with the cellular protein transport inhibitors monensin and brefeldin A, followed by analysis by flow cytometry. Note the production of IFNG by FOXP3+ CAR Tregs. PMA/Iono, Phorbol Myristate Acetate (PMA) and ionomycin treatment, a positive control for cytokine production.

FIG. 29: IL-2 production by Tregs and Teff cells activated via CAR or TCR/CD28. Intracellular staining for the Treg transcription factor FOXP3 and cytokines in cells activated overnight and then treated with the cellular protein transport inhibitors monensin and brefeldin A, followed by analysis by flow cytometry. PMA/Iono, Phorbol Myristate Acetate (PMA) and ionomycin treatment, a positive control for cytokine production.

FIG. 30: IL-3 production by Tregs and Teff cells activated via CAR or TCR/CD28. Intracellular staining for the Treg transcription factor FOXP3 and cytokines in cells activated overnight and then treated with the cellular protein transport inhibitors monensin and brefeldin A, followed by analysis by flow cytometry. PMA/Iono, Phorbol Myristate Acetate (PMA) and ionomycin treatment, a positive control for cytokine production.

FIG. 31: IL-5 production by Tregs and Teff cells activated via CAR or TCR/CD28.Intracellular staining for the Treg transcription factor FOXP3 and cytokines in cells activated overnight and then treated with the cellular protein transport inhibitors monensin and brefeldin A, followed by analysis by flow cytometry. PMA/Iono, Phorbol Myristate Acetate (PMA) and ionomycin treatment, a positive control for cytokine production.

FIG. 32: IL-17A production by Tregs and Teff cells activated via CAR or TCR/CD28.Intracellular staining for the Treg transcription factor FOXP3 and cytokines in cells activated overnight and then treated with the cellular protein transport inhibitors monensin and brefeldin A, followed by analysis by flow cytometry. PMA/Iono, Phorbol Myristate Acetate (PMA) and ionomycin treatment, a positive control for cytokine production.

FIG. 33: IFNG is produced by both CAR and TCR Teff cells, but only by CAR Tregs, as measured by intracellular protein staining. Note that CAR Treg and TCR Treg had identical FOXP3 positivity, yet CAR Tregs were IFNG positive whereas TCR Tregs were not. PMA, Phorbol Myristate Acetate (PMA) and ionomycin treatment, a positive control for cytokine production. *, p<0.05; **, p<0.01; ***, p<0.001; ****, p<0.0001, ns, not significant.

FIG. 34: Expression of transcription factor genes in Tregs activated via CAR or TCR/CD28 at the single-cell gene expression cluster level. Left-hand: Transcription factors previously implicated in the polarization and maintenance of different CD4+ T helper cell types (Th1, Th2, Th17). Right-hand: Heatmap of transcription factor gene expression across Treg single-cell gene expression gene clusters. Note that the only transcription factor gene overexpressed in cluster 9 (enriched in CAR Tregs) compared with all other clusters is HLX.

FIG. 35: Expression of TBX21, HLX, and IFNG genes in Tregs and Teff cells activated via CAR or TCR/CD28 measured by RNA sequencing.

FIG. 36: Co-expression analysis of TBX21, HLX, IRF8, and ZEB2 with IFNG in Tregs at the single-cell level. IFNG is expressed by 8.02% of analyzed Tregs at the single-cell level. IFNG expression is marked in red and transcription factor (TBX21, HLX, IRF8, or ZEB2) expression is marked in blue in the UMAP plots. ZEB2 correlates best with IFNG among the four tested transcription factor genes.

FIG. 37: Correlation of expression of IFNG with expression of all other genes in Tregs via CAR or TCR/CD28 at the single-cell level. Fifteen genes most correlated with IFNG expression in Tregs (CAR—blue, TCR—red, NoAct—black) with R-value (top) and regression curve (green).

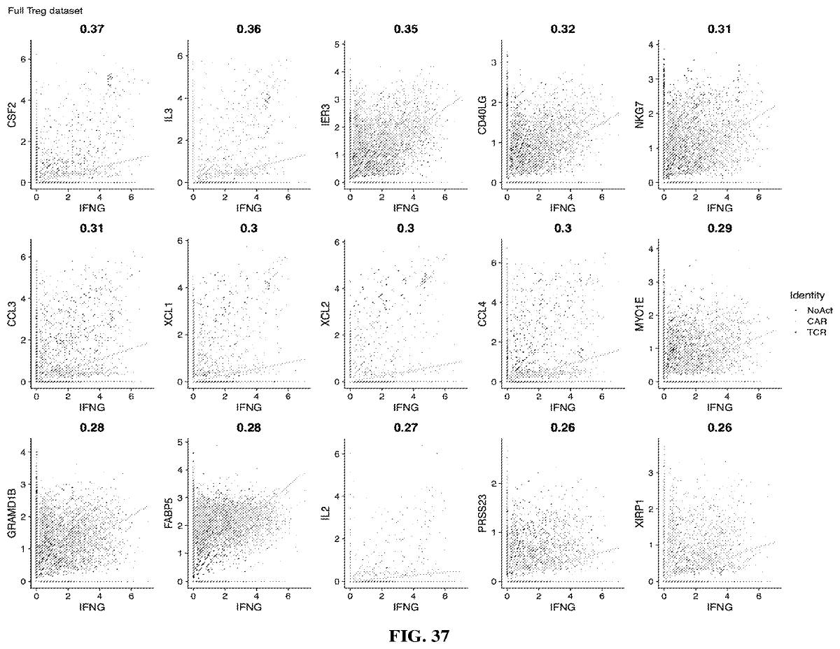

FIGS. 38A-38C: Chimeric antigen receptor signaling confers both regulatory and effector cell properties to mouse Tregs. (FIG. 38A) Mouse CD19 CAR CD4+CD25+ Tregs are cytotoxic towards mouse CD19+A20 lymphoma cells in vitro to an even greater extent than mouse CD19 CAR CD4+CD25− Tconv (conventional T cells). (FIG. 38B) Mouse CD19 CAR CD4+CD25+Tregs suppress the proliferation of CD4+CD25-Tconv cells in vitro. (FIG. 38C) Mouse CD19 CAR CD4+CD25+ Tregs express the Treg lineage transcription factor Foxp3, as assessed by intracellular protein staining.

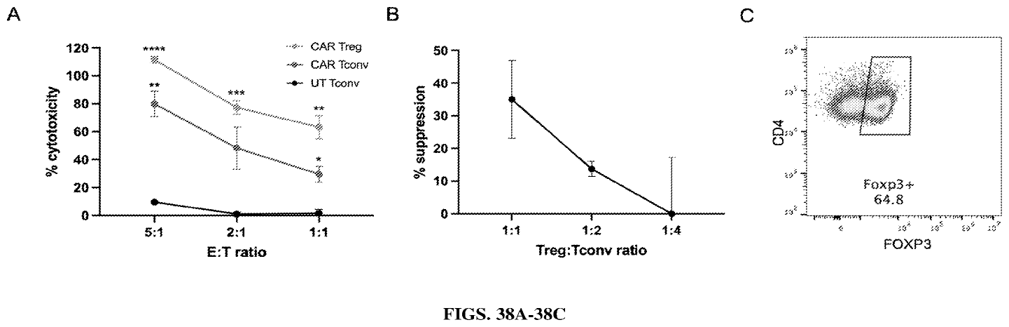

FIG. 39: CAR Tregs as a new therapy for solid tumors. The tumor microenvironment (TME) of solid tumors has immunosuppressive properties, preventing the infiltration of conventional T cells. The T cells that do infiltrate become exhausted. Cell types enriched in the TME include cancer-associated fibroblasts (CAF), tumor-associated macrophages (TAM), myeloid-derived suppressor cells (MDSC), and regulatory T cells (Treg). By engineering Tregs to recognize antigens on the surface of cancer cells with a chimeric antigen receptor (CAR), these CAR Tregs will infiltrate solid tumors and kill tumor cells via the perforin/granzymc pathway, IFN-gamma and TNF-alpha secretion, and other pathways, either directly induced by CAR activation or added on using synthetic gene circuits. The IFN-gamma and other cytokines and chemokines made by CAR Tregs can also destabilize the tumor-resident Tregs and recruit effector immune cells. Altogether, CAR Tregs can be a new therapy for solid tumors, targeting tumor cells directly and shifting the balance in the TME from anti-inflammatory to pro-inflammatory.

### DESCRIPTION OF ILLUSTRATIVE EMBODIMENTS

Regulatory T cells (T_{regs}) are a subset of T cells dedicated to suppressing immune responses. Tregs are essential to maintain self-tolerance and immune homeostasis, as dramatically illustrated by the rampant multiorgan autoimmunity observed in human patients and mice with genetic defects in FOXP3, the master transcription factor of the Treg lineage (1). Yet, T_{regs} also constitute a barrier to anticancer immunity by heavily accumulating in tumors, inhibiting their clearance by tumor antigen-specific T cells (2). Conferring antigen specificity to immune cells using a chimeric antigen receptor (CAR) dramatically expands what cells can be used and what targets can be pursued in immunotherapies. CARs are artificial receptors comprising an extracellular antigen-binding domain and an intracellular signaling domain (3). CAR T cell therapy has accomplished great success in liquid tumors, with five CD19 CAR T cell therapies for leukemia currently approved by the FDA. The same cannot be said of solid tumors: CAR T cells either fail to penetrate the tumor microenvironment or become exhausted once in it (4). Yet, thymically-derived T_{regs} migrate to solid tumors and remain abundant (2,5).

Solid tumors have revealed extremely refractory to immune-based therapies, specifically those involving T cells. This is in part because solid tumors create a tumor microenvironment that is difficult to penetrate by T cells and is replete with suppressive immune cells that lead to T cell suppression and T cell exhaustion. T_{regs}, on the other hand, have been found to traffic to tumors and thrive in its hostile microenvironment (hypoxia, glucose deprivation, lactic acid excess). The present studies found that if Tregs are redirected using a chimeric antigen receptor (CAR) specific to tumor cells, those engineered CAR Tregs kill antigen-expressing tumor cells in vitro and control their growth in vivo in preclinical humanized mouse experiments. This idea can tremendously help solid tumor patients by creating immune cells that can successfully infiltrate the solid tumor and destroy tumor cells, leading to remission in hard-to-treat cancers. Thus, the present studies focused on converting Tregs from solid tumor protectors to anti-tumor effector cells.

In the present studies, an anti-CD19 CAR with a tandem CD28-CD3ξ intracellular domain was introduced in human Tregs. Upon in vitro co-incubation with CD19₊ tumor cells, CAR T_{regs}upregulated activation markers, proliferated, secreted IL-10, and suppressed T cell proliferation, while maintaining high FOXP3 expression and a demethylated T_{reg}-specific demethylated region (TSDR). However, single-cell cytokine analysis revealed that CAR-mediated activation of Tregs also leads to high production of the inflammatory and cytolytic molecules IFN-γ, TNF-α, perforin, and granzyme B. Strikingly, CAR T_{regs} suppressed CD19⁺ tumor cell growth in NSG mice. This phenomenon was observed across three tumor cell types (B-cell leukemia, myeloid leukemia, and epithelial carcinoma) and two routes of delivery (intravenous and subcutaneous). Annexin V staining and quantitation of tumor cells co-incubated with CAR T_{regs} in vitro confirmed that CD19 CAR Tregs kill CD19+ tumor cells of different origins. It was hypothesized that CAR signaling, originally designed to trigger inflammatory cytokine secretion and target cell killing by CAR T cells in cancer therapy, exacerbates human T_{reg} cytotoxicity, potentially transforming them into a novel solid cancer therapy. Surprisingly, regulatory T cells, seen as suppressive T cells enriched in solid tumors that thwart anti-tumor responses and are a poor prognostic indicator, can be used to eradicate solid tumors. Thus, in certain embodiments, the present disclosure provides methods for treating cancer by CAR-induced T_{reg}-mediated cytotoxicity.

Some conventional T cells can recognize tumor cells, but either fail to penetrate the solid tumor microenvironment or become exhausted once in there. This is due to a mix of harsh conditions (low oxygen, low glucose, high lactic acid) and suppressive cells (myeloid-derived suppressor cells, regulatory T cells, tumor cells themselves express PD-L1, an exhaustion-inducing molecule). T_{regs} cells, in contrast, migrate to solid tumors thanks to their chemokine receptor expression patterns and thrive there, thanks to their ability to take in lactic acid and use it as fuel, for example. T_{regs} are HLA class II-restricted, and HLA class II is only expressed in professional antigen presenting cells, so Tregs do not even see tumor cells directly, as they usually only express HLA class I and maybe even at low levels (HLA downregulation typical tumor evasion strategy). But with a CAR in T_{regs} specific for a cancer cell, the T_{regs} can be forced to directly attach to a tumor cell with high affinity and kill the tumor cells they recognize. In further aspects, the T_{regs} can be engineered to deliver toxic payloads to cancer cells.

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “about” means, in general, within a standard deviation of the stated value as determined using a standard analytical technique for measuring the stated value. The terms can also be used by referring to plus or minus 5% of the stated value.

The phrase “effective amount” or “therapeutically effective” means a dosage of a drug or agent sufficient to produce a desired result. The desired result can be subjective or objective improvement in the recipient of the dosage, increased lung growth, increased lung repair, reduced tissue edema, increased DNA repair, decreased apoptosis, a decrease in tumor size, a decrease in the rate of growth of cancer cells, a decrease in metastasis, or any combination of the above.

As used herein, the term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. The antibody may be a bi-specific antibody. In exemplary embodiments, antibodies used with the methods and compositions described herein are derivatives of the IgG class. The term antibody also refers to antigen-binding antibody fragments. Examples of such antibody fragments include, but are not limited to, Fab, Fabÿ, F(abÿ)2, scFv, Fv, d_{S}Fv diabody, and Fd fragments. Antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 10 amino acids and more typically will comprise at least about 200 amino acids.

“Subject” and “patient” refer to either a human or non-human, such as primates, mammals, and vertebrates. In particular embodiments, the subject is a human.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

The term “T cell” refers to T lymphocytes as defined in the art and is intended to include thymocytes, immature T lymphocytes, mature T lymphocytes, resting T lymphocytes, or activated T lymphocytes. The T cells can be CD4⁺ T cells, CD8⁺ T cells, CD4⁺CD8⁺T cells, or CD4⁻CD8⁻ cells. The T cells can also be T helper cells, such as T helper 1 (TH1), or T helper 2 (TH2) cells, or TH17 cells, as well as cytotoxic T cells, regulatory T cells, natural killer T cells, naïve T cells, memory T cells, or gamma delta T cells (Wilson et al., 2009; Wynn, 2005; Ladi et al., 2006). T cells that differ from each other by at least one marker, such as CD4, are referred to herein as “subsets” of T cells.

“CD4+ T cells” refers to a subset of T cells that express CD4 on their surface and are associated with cell-mediated immune response. They are characterized by the secretion profiles following stimulation, which may include secretion of cytokines such as IFN-gamma, TNF-alpha, IL-2, IL-4 and IL-10. “CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8⁺ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

“Regulatory T cells” refer to a subset of T cells which act to suppress immune responses, thereby maintaining homeostasis and self-tolerance. Self-tolerance refers to a state of immune unresponsiveness towards self-antigens, important to avoid the development of autoimmune disease.

The term “chimeric antigen receptors (CARs),” as used herein, may refer to artificial T cell receptors, chimeric T cell receptors, or chimeric immunoreceptors, for example, and encompass engineered receptors that graft an artificial specificity onto a particular immune effector cell. CARs may be employed to impart the specificity of a monoclonal antibody onto a T cell, thereby allowing a large number of specific T cells to be generated, for example, for use in adoptive cell therapy. In specific embodiments, CARs direct specificity of the cell to a tumor associated antigen, for example. In some embodiments, CARs comprise an intracellular activation domain, a transmembrane domain, and an extracellular domain comprising a tumor associated antigen binding region. In particular aspects, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins. In certain cases, the spacing of the antigen-recognition domain can be modified to reduce activation-induced cell death. In certain cases, CARs comprise domains for additional co-stimulatory signaling, such as CD3ζ, FcR, CD27, CD28, CD137, DAP10, and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

The term “antigen presenting cells (APCs)” refers to a class of cells capable of presenting one or more antigens in the form of peptide-MHC complex recognizable by specific effector cells of the immune system, and thereby inducing an effective cellular immune response against the antigen or antigens being presented. APCs can be intact whole cells such as macrophages, B cells, endothelial cells, activated T cells, and dendritic cells; or other molecules, naturally occurring or synthetic, such as purified MHC Class I molecules complexed to β2-microglobulin. While many types of cells may be capable of presenting antigens on their cell surface for T cell recognition, only dendritic cells have the capacity to present antigens in an efficient amount to activate naive T cells for cytotoxic T lymphocyte (CTL) responses.

### II. CAR-Modified T Cells

In certain embodiments, the present disclosure provides T_{regs} engineered to express a CAR vector. The CAR T_{regs} may be used to treat a disease or disorder, such as a solid tumor or blood cancer.

Certain embodiments of the present disclosure concern obtaining a starting population of T_{regs}, modifying the T_{regs}, and administering the modified T_{regs} to a subject as an immunotherapy to target cancer cells. In particular, the T_{regs} express CAR.

In some embodiments, the starting population of T_{regs} are derived from the blood, bone marrow, lymph, or lymphoid organs. In some aspects, the cells are human cells. The cells typically are primary cells, such as those isolated directly from a subject and/or isolated from a subject and frozen. With reference to the subject to be treated, the cells may be allogeneic and/or autologous. In some embodiments, the methods include isolating cells from the subject, preparing, processing, culturing, and/or engineering them, as described herein, and re-introducing them into the same patient, before or after cryopreservation.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., regulatory T cells) but are present or expressed at relatively higher levels on certain other populations of T cells (e.g., regulatory T cells).

In some embodiments, regulatory T cells are separated from a PBMC sample by negative selection of markers expressed on non-T cells, such as B cells, monocytes, or other white blood cells, such as CD14. In some aspects, CD4+CD25+CD127− Tregs are purified from human peripheral blood.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days. For example, the cells may be cultured from 5 days, 5.5 days, or 5.8 days to 21 days, 21.5 days, or 21.8 days, such as from 10 days, 10.5days, or 10.8 days to 14 days, 14.5 days, or 14.8 days.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days. Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15). The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as a human leukocyte antigen A2 (HLA-A2) binding peptide, in the presence of a T-cell growth factor, such as 300 IU/ml IL-2. The in vitro-induced T-cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2⁺ allogeneic lymphocytes and IL-2, for example.

The autologous T-cells can be modified to express a T-cell growth factor that promotes the growth and activation of the autologous T-cells. Suitable T-cell growth factors include, for example, interleukin (IL)-2, IL-7, IL-15, and IL-12. Suitable methods of modification are known in the art. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994. In particular aspects, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression.

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

In some embodiments, the CAR contains an extracellular antigen-recognition domain that specifically binds to an antigen. In some embodiments, the antigen is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Patent Nos.: 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLOS ONE 8(4): e61338; Turtle et al., Curr. Opin. Immunol., 2012 October; 24(5): 633-39; Wu et al., Cancer, 2012 Mar. 18(2): 160-75. In some aspects, the genetically engineered antigen receptors include a CAR as described in U.S. Patent No.: 7,446,190, and those described in International Patent Application Publication No.: WO/2014055668 A1.

In some embodiments, the CAR comprises: a) an intracellular signaling domain, b) a transmembrane domain, and c) an extracellular domain comprising an antigen binding region.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the invention includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, and 4-1BB (CD137). In addition to a primary signal initiated by CD3ζ, an additional signal provided by a human costimulatory receptor inserted in a human CAR is important for full activation of NK cells and could help improve in vivo persistence and the therapeutic success of the adoptive immunotherapy.

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a tumor associated antigen or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. Exemplary embodiments of tumor associated antigens include CD19 CD20, carcinoembryonic antigen, alphafetoprotein, CA-125, MUC-1, CD56, EGFR, c-Met, AKT, Her2, Her3, epithelial tumor antigen, melanoma-associated antigen, mutated p53, mutated ras, and so forth.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimcric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-ζ, CD137/CD3-ζ, or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CAR⁺ immune cells (Singh et al., 2008; Singh et al., 2011).

Among the target proteins of antigens targeted by the present CARs are those expressed in the context of a disease, condition, or cell type to be targeted via the CAR. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells. Any suitable antigen may find use in the present methods. Exemplary antigens include, but are not limited to, antigenic molecules from infectious agents, auto-/self-antigens, tumor-/cancer-associated antigens, and tumor neoantigens.

The terms “tumor-associated antigen,” “tumor antigen” and “cancer cell antigen” are used interchangeably herein. In each case, the terms refer to proteins, glycoproteins or carbohydrates that are specifically or preferentially expressed by cancer cells.

A tumor associated antigen may be of any kind so long as it is expressed on the cell surface of tumor cells. Tumor-associated antigens may be derived from prostate, breast, colorectal, lung, pancreatic, renal, mesothelioma, ovarian, sarcoma or melanoma cancers. Exemplary tumor-associated antigens or tumor cell-derived antigens include MAGE 1, 3, and MAGE 4 (or other MAGE antigens such as those disclosed in International Patent Publication No. WO99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Exemplary embodiments of tumor associated antigens include, but are not limited to, CD19, CD20, carcinoembryonic antigen, alphafetoprotein, CA-125, MUC-1, CD56, EGFR, c-Met, AKT, Her2, Her3, epithelial tumor antigen, melanoma-associated antigen, mutated p53, mutated ras, and so forth. In particular aspects, the antigens include NY-ESO, EGFRvIII, Muc-1, Her2, CA-125, WT-1, Mage-A3, Mage-A4, Mage-A10, TRAIL/DR4, and CEA. In particular aspects, the antigens for the two or more antigen receptors include, but are not limited to, CD19, EBNA, WT1, CD123, NY-ESO, EGFRvIII, MUC1, HER2, CA-125, WT1, Mage-A3, Mage-A4, Mage-A10, TRAIL/DR4, and/or CEA. The sequences for these antigens are known in the art, for example, CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUCI (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4,MAGE-A6, MAGE-A10, MAGE-A12, MART-1, BAGE, DAM-6, -10, GAGE-1, -2, -8,GAGE-3, -4, -5, -6, -7B, NA88-A, MART-1, MC1R, Gp100, PSA, PSM, Tyrosinase, TRP-1,TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP,-catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2,707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX

(CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAGIB, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

### A. Antigen-Presenting Cells

APCs, which include macrophages, B lymphocytes, and dendritic cells, are distinguished by their expression of a particular MHC molecule. APCs internalize antigen and re-express a part of that antigen, together with the MHC molecule on their outer cell membrane. The MHC is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is referred to as the HLA complex and in mice the H-2 complex.

In some cases, aAPCs are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662.

aAPC systems may comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD86, CD64 (FcγRI), 41BB ligand, and IL-21. Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), which promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001.

### B. Formulation and Administration

The present disclosure provides pharmaceutical compositions comprising CAR T_{regs}. Such compositions comprise a prophylactically or therapeutically effective amount of T_{regs} and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Generally, the ingredients of compositions of the disclosure are supplied either separately or mixed together in unit dosage form, for example, as a dry lyophilized powder or water-free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### C. Hyperproliferative Diseases

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases.

Cancer cells that may be treated with the compounds of the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; lciomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### D. Methods of Treatment

In particular, compositions that may be used in treating cancer in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells or killing bacterial cells). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that inhibits the growth or proliferation of a bacterial cell, inhibits the growth of a biofilm, or induces death of cancerous cells (e.g., induces apoptosis of a cancer cell), as assayed by identifying a reduction in hematological parameters (Complete blood count (CBC)), or cancer cell growth or proliferation.

In some embodiments, the present disclosure provides methods for immunotherapy comprising administering an effective amount of the T_{regs} of the present disclosure. In one embodiment, a medical disease or disorder is treated by transfer of an immune cell population that elicits an immune response. In certain embodiments of the present disclosure, cancer is treated by transfer of a T_{reg} population that elicits an immune response. Provided herein are methods for treating or delaying progression of cancer in an individual comprising administering to the individual an effective amount an antigen-specific cell therapy. The present methods may be applied for the treatment of immune disorders, solid cancers, hematologic cancers, and viral infections.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp;

adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mcsoncphroma, malignant; hemangiosarcoma; hcmangiocndothclioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma;

neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

To determine the suitability of cells provided herein for therapeutic applications, the cells can first be tested in a suitable animal model. At one level, cells are assessed for their ability to survive and maintain their phenotype in vivo. Cells provided herein are administered to immunodeficient animals (such as NOG mice, or animals rendered immunodeficient chemically or by irradiation) at a site amenable for further observation, such as under the kidney capsule, into the spleen, into a liver lobule, or into the bone marrow. Tissues are harvested after a period of a few days to several weeks or more, and assessed as to whether starting cell types such as erythrocytes are still present. This can be performed by providing the administered cells with a detectable label (such as green fluorescent protcin, or β-galactosidasc); or by measuring a constitutive marker specific for the administered human cells. Where cells provided herein are being tested in a rodent model, the presence and phenotype of the administered cells can be assessed by immunohistochemistry or ELISA using human-specific antibody, or by RT-PCR analysis using primers and hybridization conditions that cause amplification to be specific for human polynucleotide sequences. Suitable markers for assessing gene expression at the mRNA or protein level are provided elsewhere in this disclosure.

T_{regs} provided by methods of the present disclosure may be tested in various animal models for their ability to treat hematological disorders and injuries. For example, a sickle cell anemia mouse model or the T/B cell-deficient Rag-2 knockout mouse may be particularly useful animal models for testing the myeloid and lymphoid cells disclosed herein.

T_{regs} provided in certain aspects of the present disclosure that demonstrate desirable functional characteristics or efficacy in animal models, may also be suitable for direct administration to human subjects in need thereof. For purposes of hemostasis, the cells can be administered at any site that has adequate access to the circulation. Hematopoietic cells or precursors thereof may also be delivered at a site of injury or disease.

The T_{regs} provided in certain aspects of this present disclosure can be used for therapy of any subject in need thereof. Human conditions that may be appropriate for such therapy include the various anemias and hemoglobinopathies, as well as diseases characterized by decreased numbers of hematopoietic cells (such as, for example, myelodysplastic syndrome, myelofibrosis, neutropenia, agranulocytosis, Glanzmann's thrombasthenia, thrombocytopenia, and acquired immune deficiency syndrome). For human therapy, the dose is generally between about 10⁹ and 10¹² cells, and typically between about 5×10⁹ and 5×10¹⁰ cells, making adjustments for the body weight of the subject, nature and severity of the affliction, and the replicative capacity of the administered cells. The ultimate responsibility for determining the mode of treatment and the appropriate dose lies with the managing clinician.

Therapeutically effective amounts of T_{regs} can be administered by a number of routes, including parenteral administration, for example, intravenous, intraperitoneal, intramuscular, intrasternal, or intraarticular injection, or infusion.

The therapeutically effective amount of T_{regs} for use in adoptive cell therapy is that amount that achieves a desired effect in a subject being treated. For instance, this can be the amount of T_{regs} necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The T_{regs} population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of T_{regs} will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of immune cells is readily determined by one of skill in the art based on the age, weight, sex, and physiological condition of the subject. Effective doses can be extrapolated from dose-response curves derived from in vitro or animal model test systems.

The T_{regs} may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acctylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In one embodiment, the disclosure provides a method of monitoring treatment progress. The method includes the step of determining a level of changes in hematological parameters and/or cancer stem cell (CSC) analysis with cell surface proteins as diagnostic markers (which can include, for example, but are not limited to CD34, CD38, CD90, and CD117) or diagnostic measurement (e.g., screen, assay) in a subject suffering from or susceptible to a disorder or symptoms thereof associated with cancer (e.g., leukemia) in which the subject has been administered a therapeutic amount of a composition as described herein. The level of marker determined in the method can be compared to known levels of marker either in healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

### E. Additional Therapy

In certain embodiments, the compositions and methods of the present embodiments involve T_{regs}, in combination with a second or additional therapy.

In certain embodiments, the compositions and methods of the present embodiments involve T_{regs} in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

An inhibitory antibody may be administered before, during, after, or in various combinations relative to an anti-cancer treatment. The administrations may be in intervals ranging from concurrently to minutes to days to weeks. In embodiments where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

Various combinations may be employed. For the example below a T_{regs} composition, is “A” and an additional anti-cancer therapy is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### 1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calichcamicin gammalI and calichcamicin omegal1); dynamicin, including dyncmicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

### 2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. No. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3to 4 wk), to single doses of 2000 to 6000 roentgens. Dosage ranges for radioisotopes vary widely, and depend on the half-life of the isotope, the strength and type of radiation emitted, and the uptake by the neoplastic cells.

### 3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immunc effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen (Carter et al., 2008; Teicher 2014; Lcal et al., 2014). Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach (Teicher 2009) and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. No. 5,801,005 and 5,739,169; Hui and Hashimoto, 1998; Christodoulides et al., 1998); cytokine therapy, e.g., interferons alpha, beta, lambda, and gamma, IL-1, GM-CSF, and TNF (Bukowski et al., 1998; Davidson et al., 1998; Hellstrand et al., 1998);

gene therapy, e.g., TNF, IL-1, IL-2, and p53 (Qin et al., 1998; Austin-Ward and Villaseca, 1998; U.S. Pat. No. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application Nos. US20140294898, US2014022021, and US20110008369, all incorporated herein by reference.

In some embodiments, the PD-1 binding antagonist is an anti-PD-1 antibody (e.g., a human antibody, a humanized antibody, or a chimcric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Patent No. U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WOO 1/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Patent Nos. 5,844,905, 5,885,796 and International Patent Application Nos. WO1995001994 and WO1998042752; all incorporated herein by reference, and immunoadhesions such as described in U.S. Pat. No. 8,329,867, incorporated herein by reference.

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### III. Kits

In various aspects of the embodiments, a kit is envisioned containing therapeutic agents and/or other therapeutic and delivery agents. In some embodiments, the present embodiments contemplates a kit for preparing and/or administering a T cell composition of the embodiments. The kit may comprise one or more sealed vials containing any of the pharmaceutical compositions of the present embodiments. The kit may include, for example, T cells as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### IV. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Generation and Characterization of CD19-CAR T_{regs}

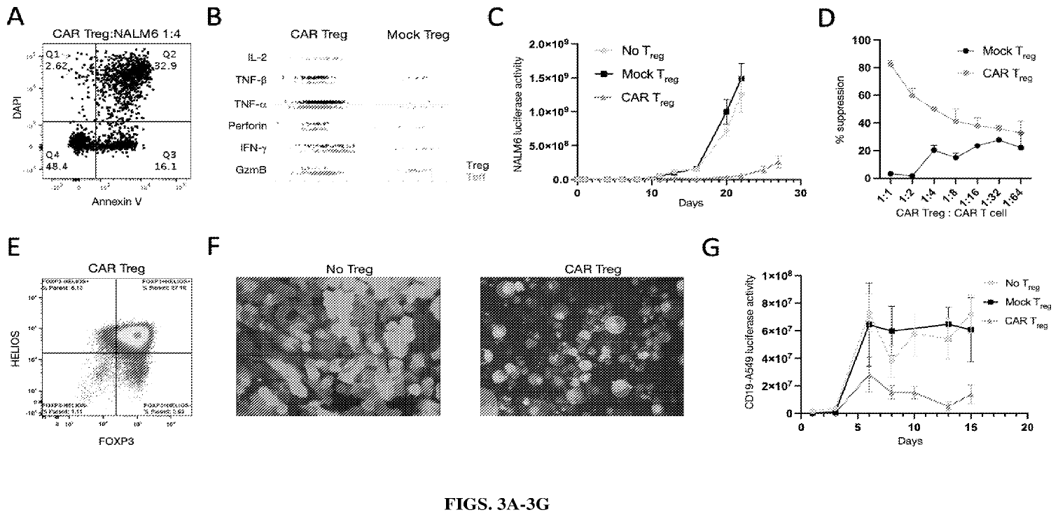

Anti-CD19 CD28-CD38 CAR T_{regs} were generated by transducing purified human peripheral blood T_{regs} with lentiviral particles containing the CAR construct. The resultant CD19CAR T_{regs} became activated and proliferated, while maintaining high FOXP3 expression (FIG. 3E) and a demethylated Treg-specific demethylated region (TSDR), in response to irradiated CD19-expressing K562 cells (a myeloid leukemia cell line devoid of HLA or CD80/86 expression), but not parental K562 cells. Surprisingly, CAR T_{regs} also efficiently killed CD19-expressing tumor cells in vitro, including NALM6 (B-cell leukemia cell line, FIG. 3A), CD19-K562, and CD19-A549 (epithelial lung cancer cell line, FIG. 3F), as assessed by Annexin V and DAPI staining and microscopy. This killing activity was contact-dependent, as co-incubating CAR T_{regs}simultaneously with differentially labeled WT NALM6 and CD19_{KO} NALM6 resulted in killing of WT NALM6 only. Surprisingly, single-cell cytokine analysis revealed that CAR-mediated activation of T_{regs} led to a cytokine production profile almost identical to that of CAR T effector (Teff) cells, i.e., abundant production of the inflammatory and cytolytic molecules IFN-γ, TNFα, perforin, and granzyme B (FIG. 3B). Moreover, CAR T_{regs} suppressed CD19₊ tumor cell growth in NSG mice when co-injected with the tumor cells, as seen in FIG. 3C for NALM6 and FIG. 3G for CD19-A549 (similar results with CD19-K562). Nonetheless, CD19 CAR T_{regs} suppressed the proliferation of CAR T_{eff} cells in a CAR-dependent manner in vitro (FIG. 3D), indicating that these modified T_{regs} retain phenotypic and functional characteristics of T cells in addition to their de novo tumor cytotoxic properties.

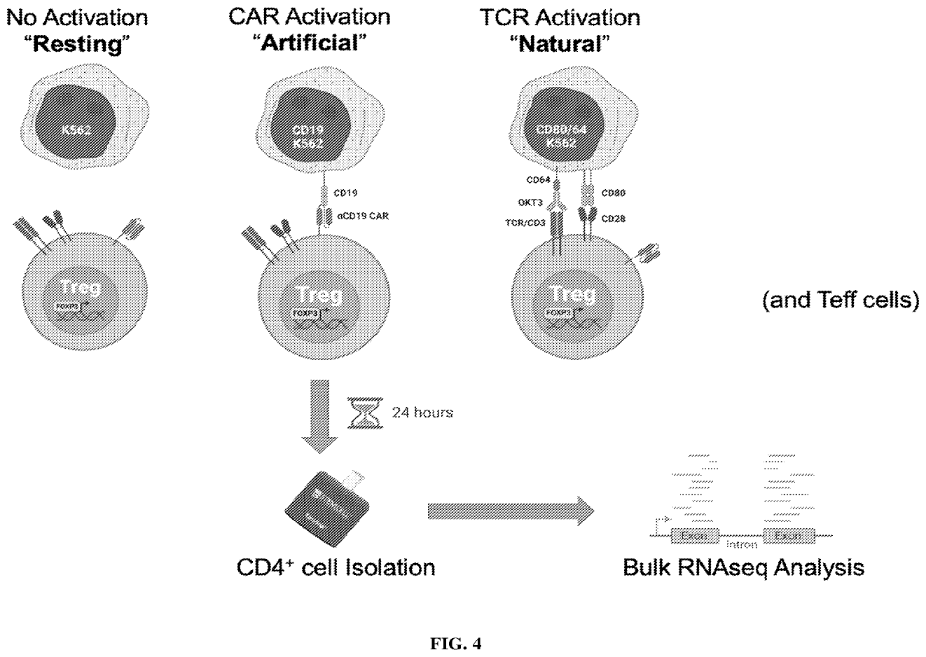

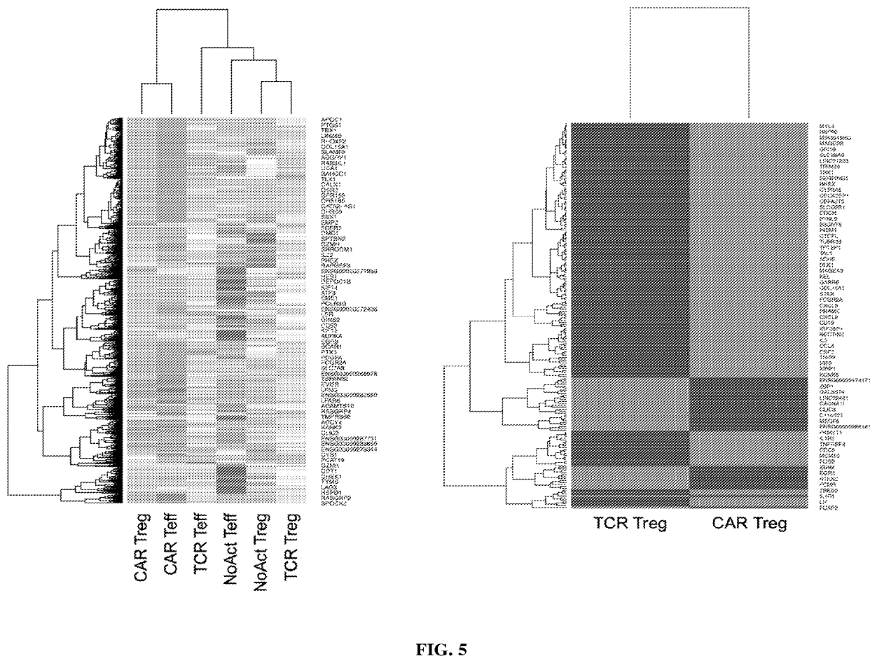

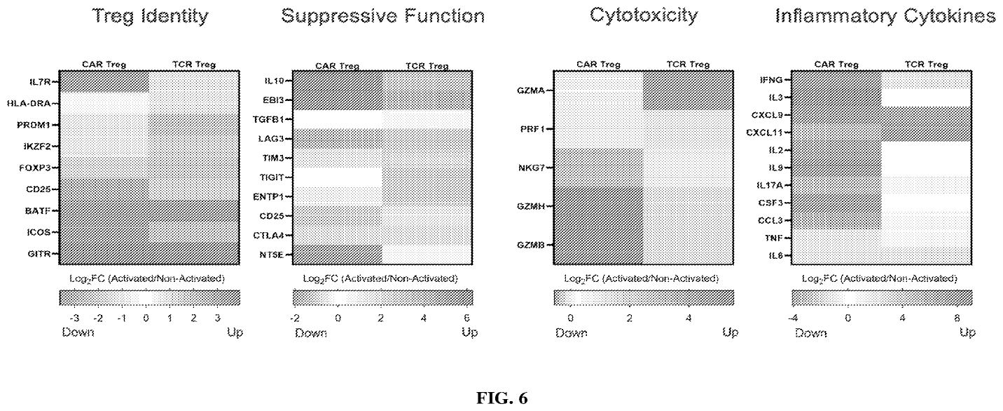

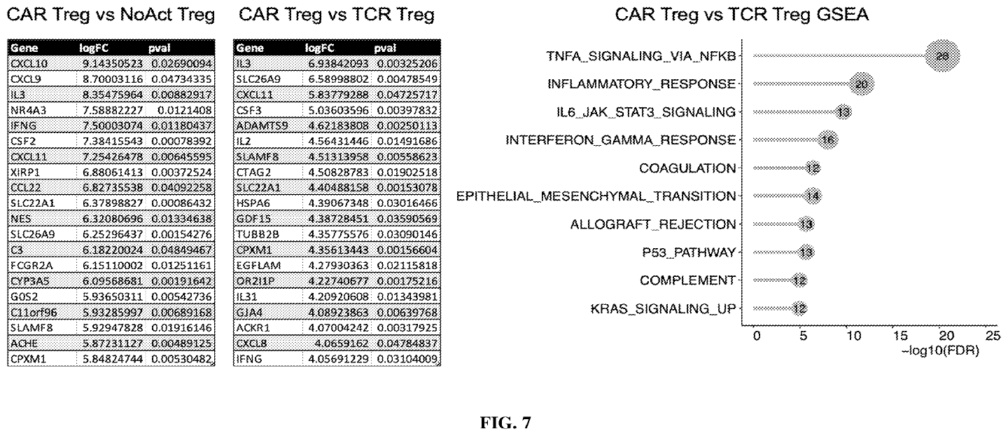

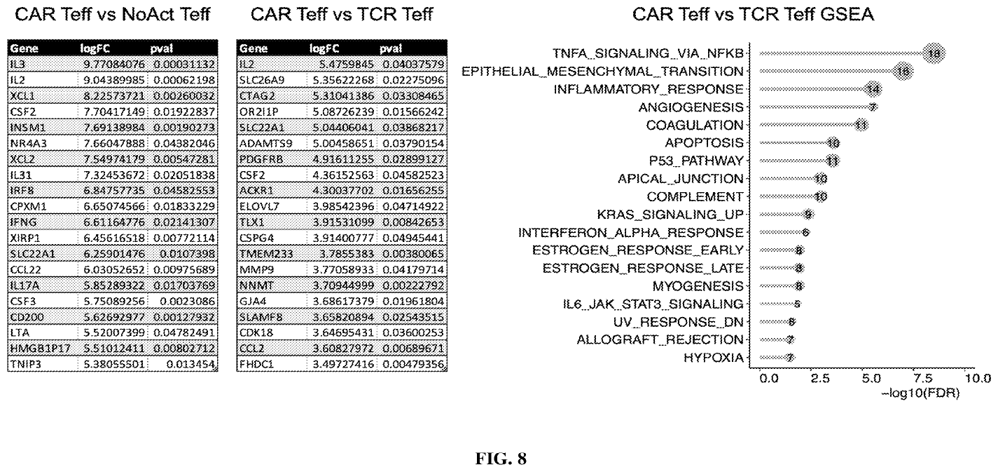

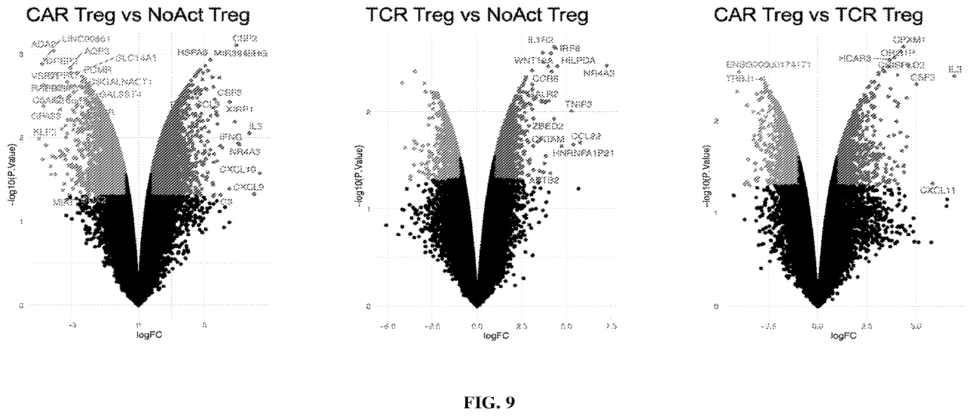

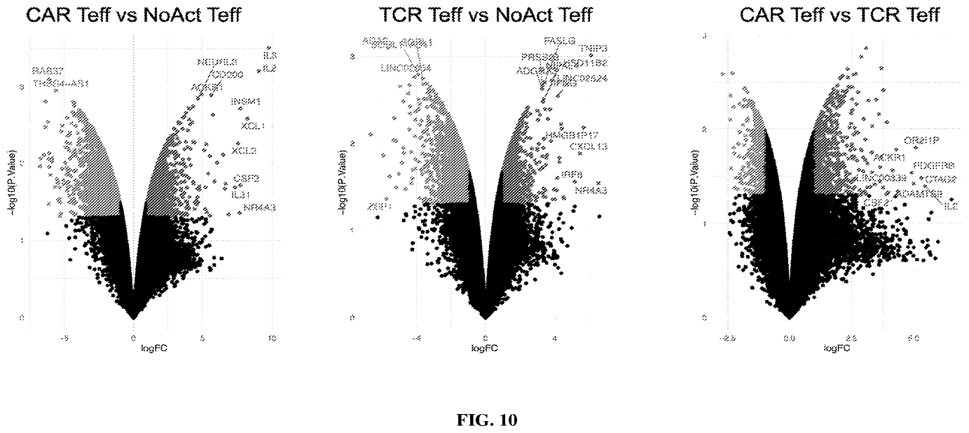

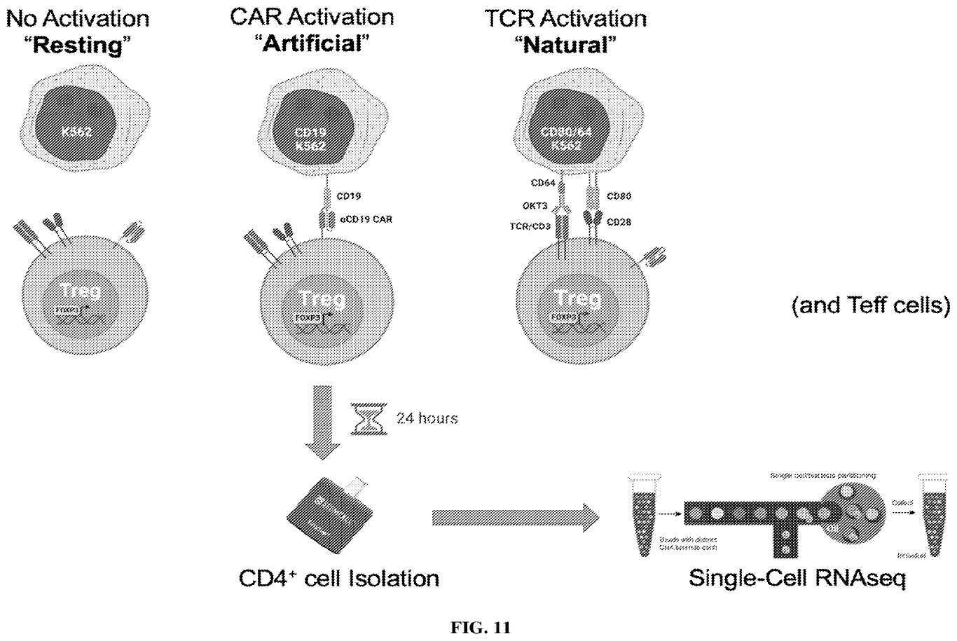

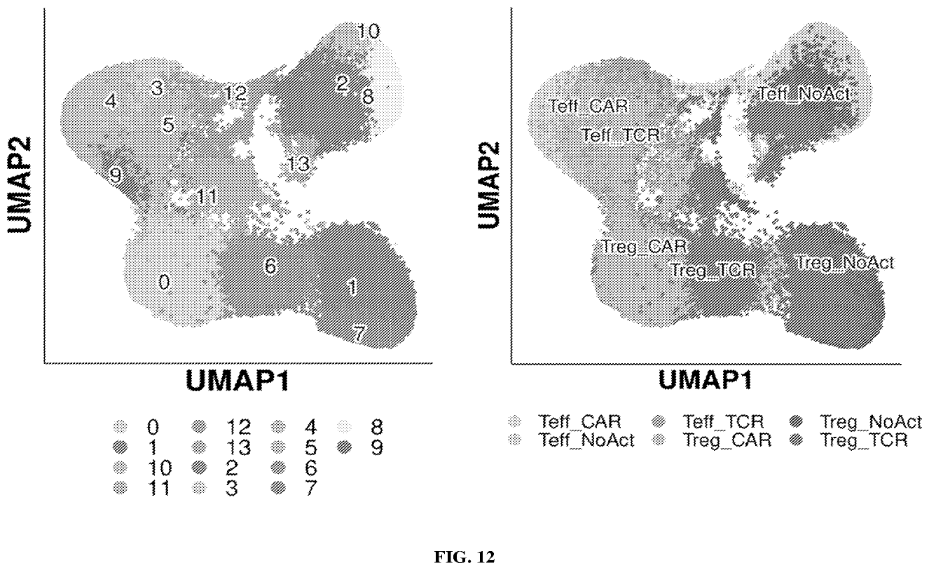

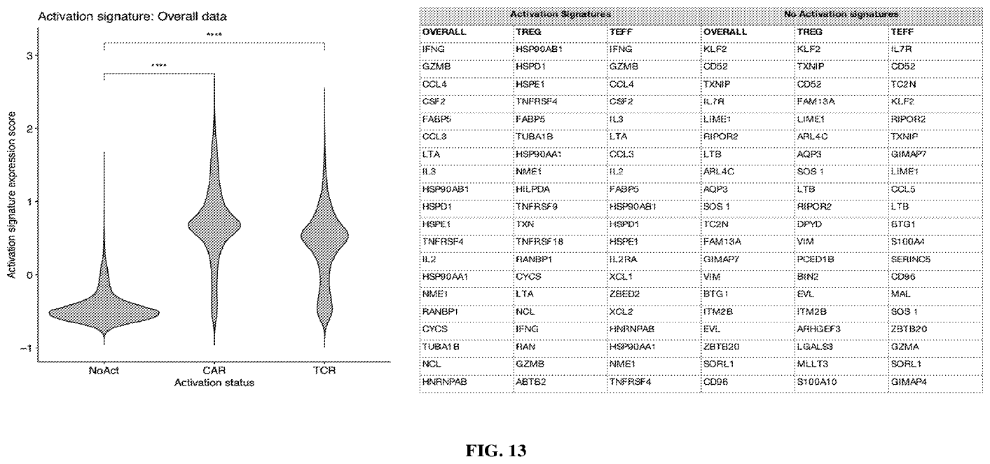

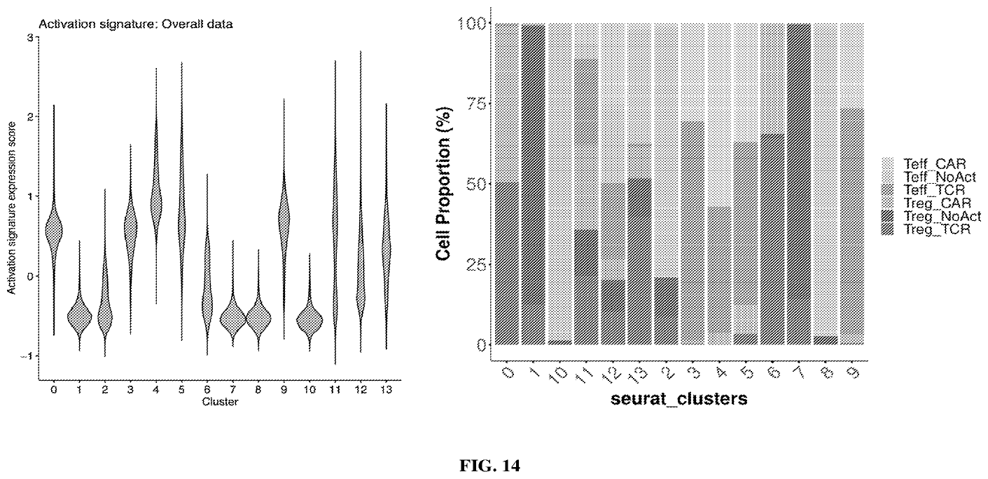

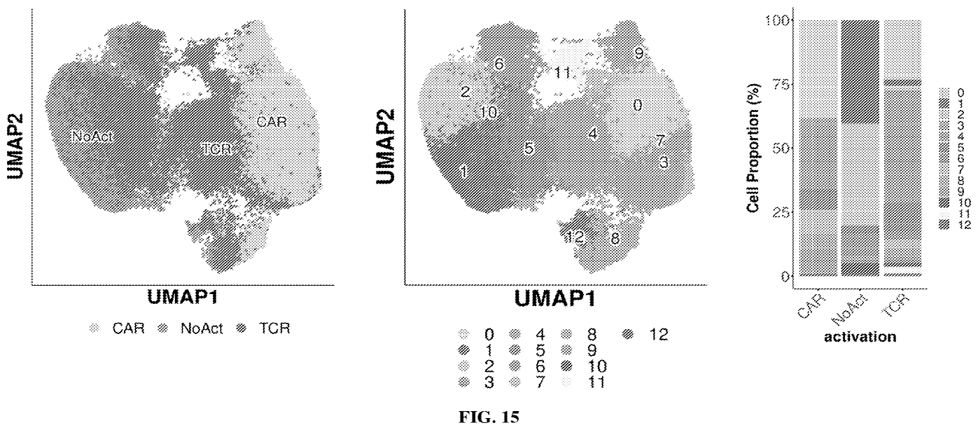

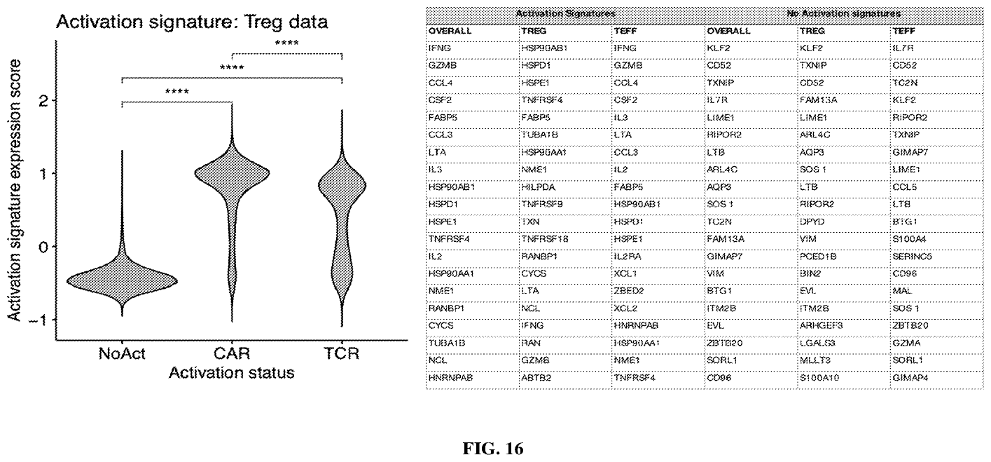

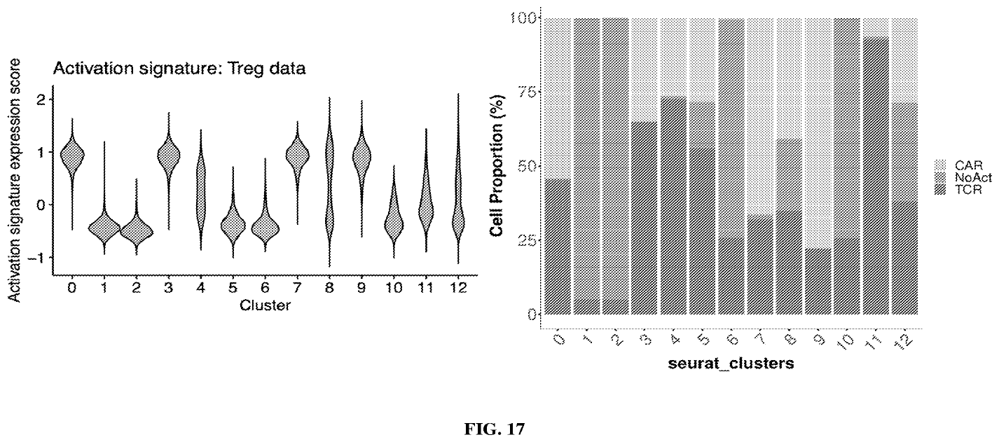

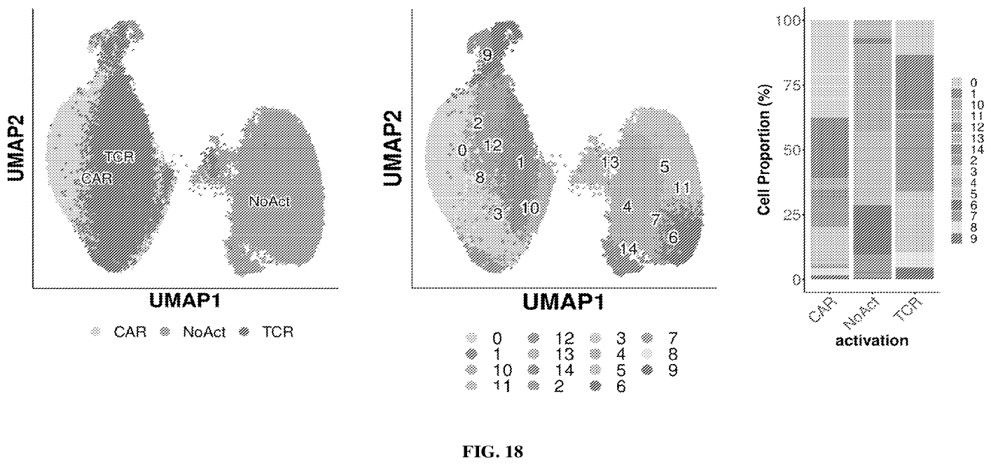

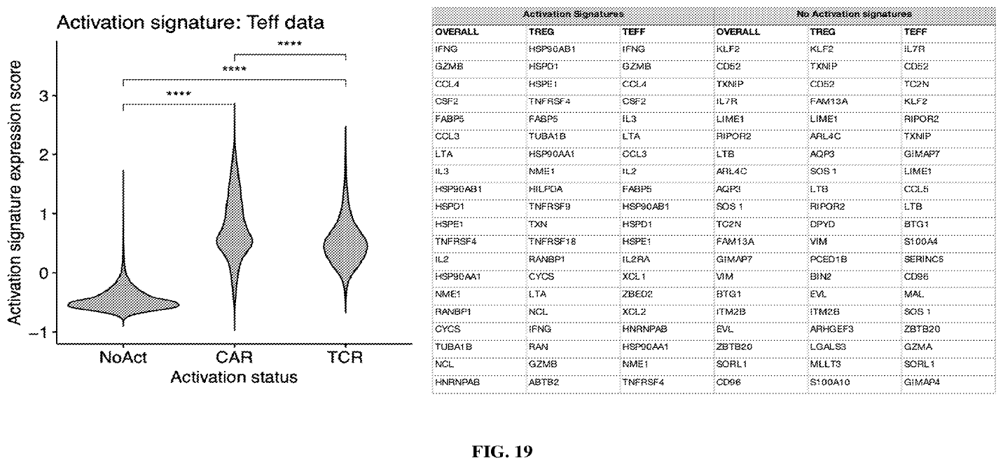

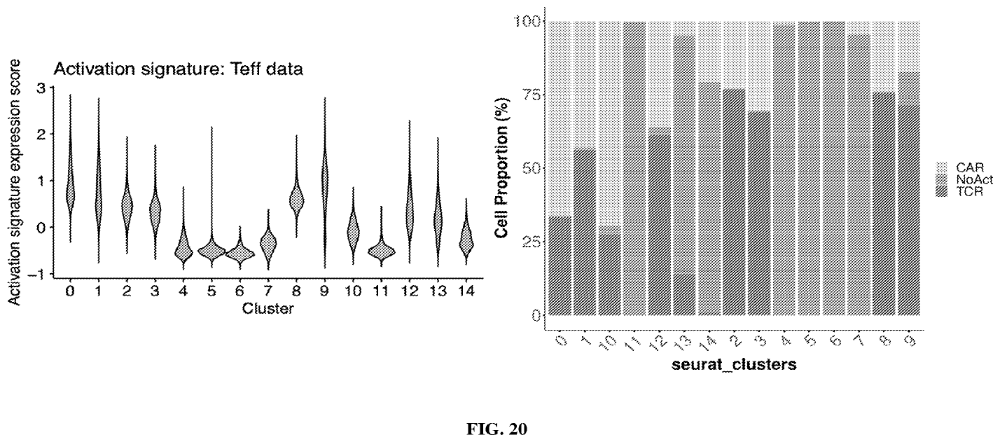

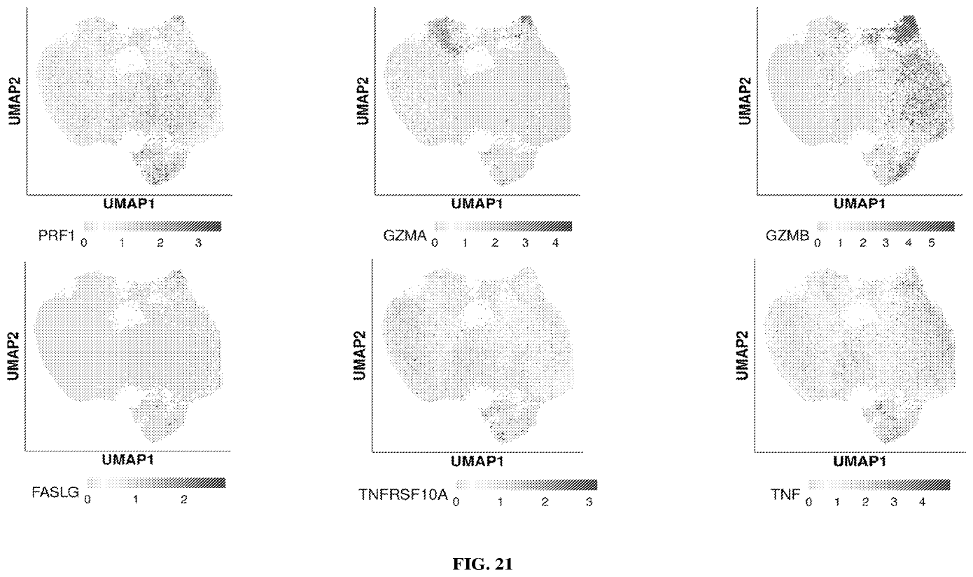

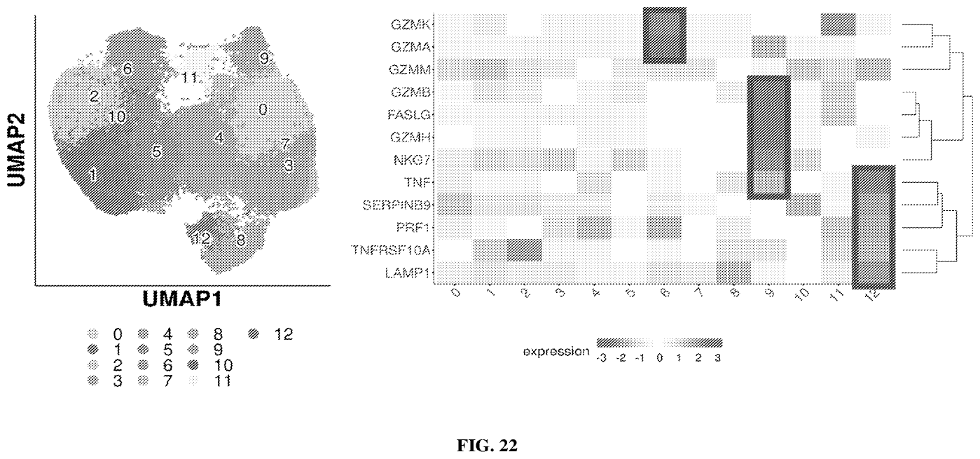

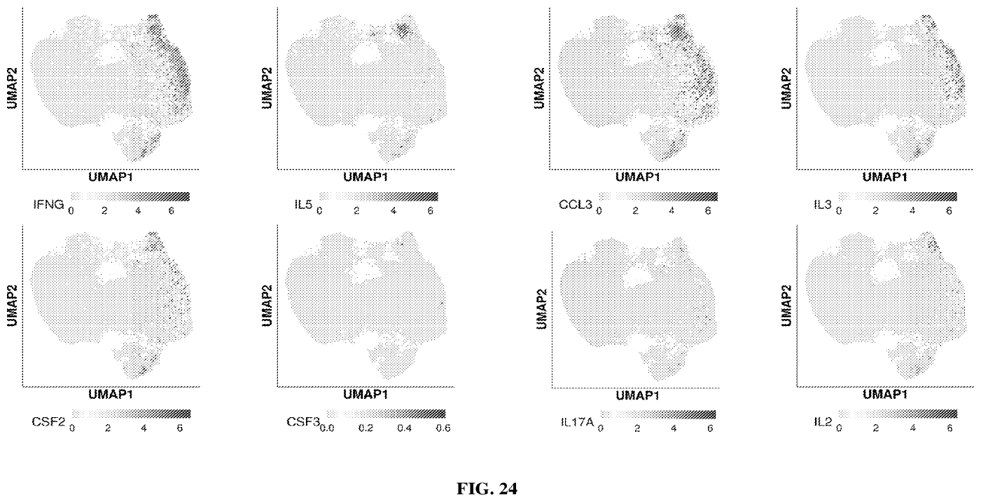

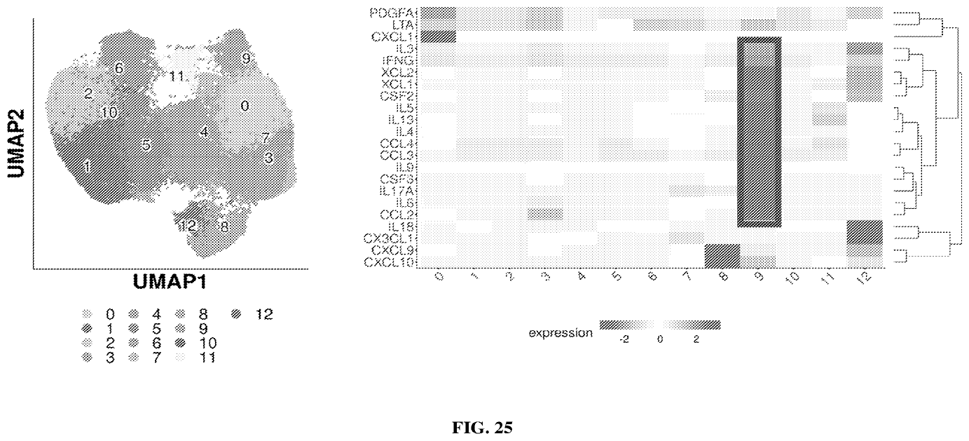

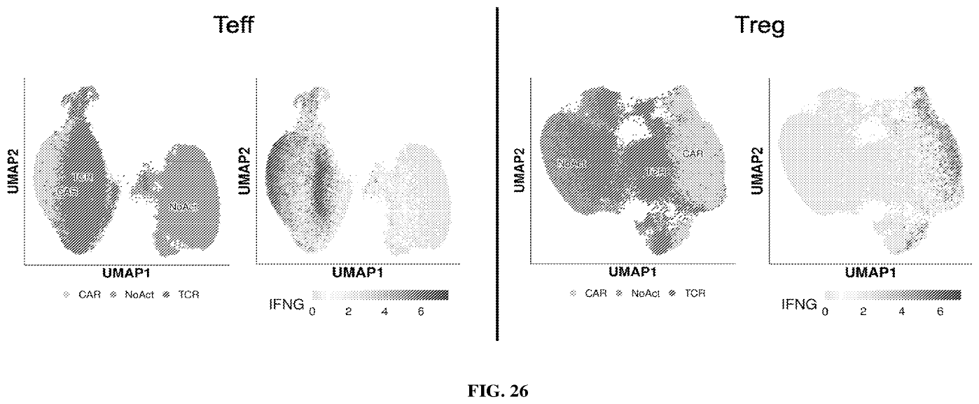

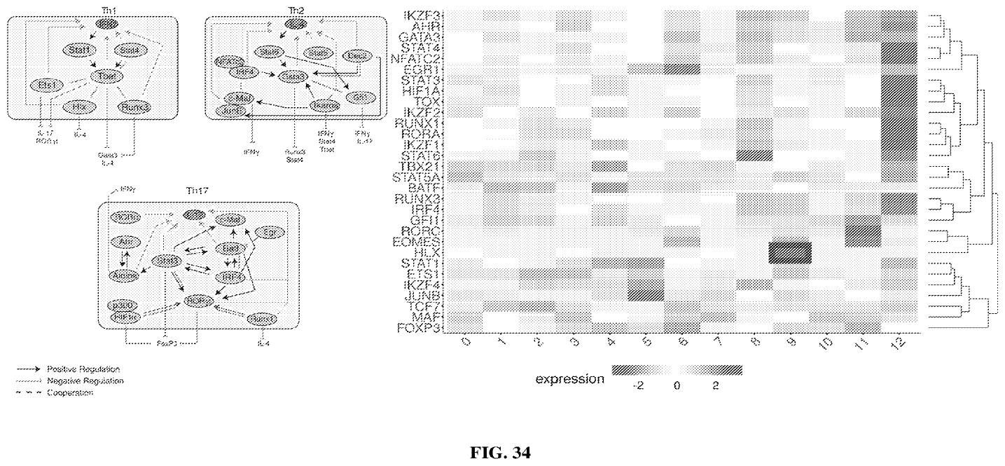

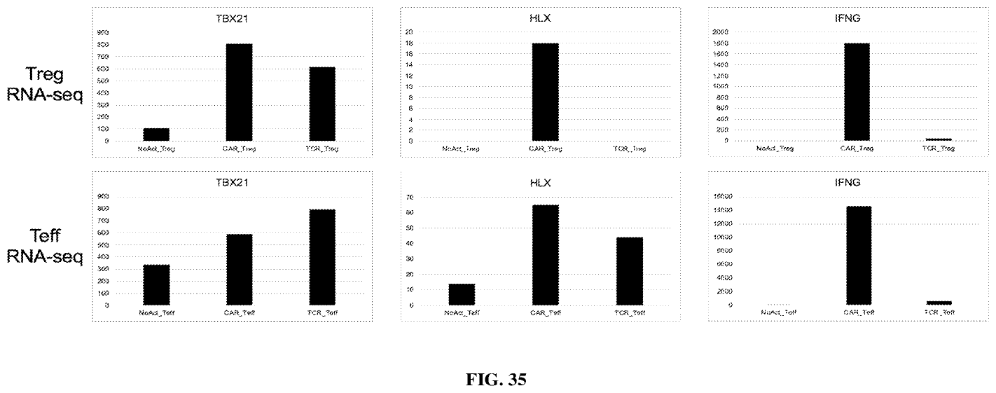

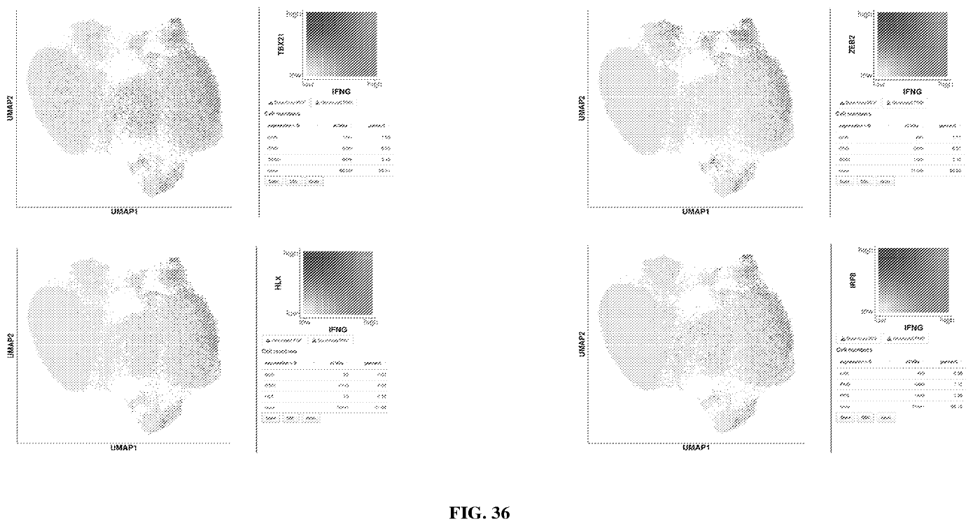

Peripheral blood mononuclear cells (PBMC) were isolated using a Ficoll gradient, CD4+ T cells were magnetically enriched, and CD4+CD25highCD127low Tregs and CD4+CD25lowCD127high Teff cells were sorted using fluorescence-assisted cell sorting (FACS). After sorting, cells were plated and activated using anti-CD3/28 beads and recombinant human IL-2. Two days later, cells were transduced with anti-CD19 CD28-CD3ζ CAR-containing lentiviral particles and expanded in the presence of IL-2 for one week. Then, CAR Tregs and CAR Teff cells were incubated with irradiated target cells, either parental K562 cells (no stimulation), CD64-CD80-K562 cells decorated with anti-CD3 antibody (TCR/CD28 stimulation), or CD19-K562 cells (CD28-CD35 CAR stimulation). CD64 is a high affinity Fc receptor; it has been previously shown that CD64-expressing K562 cells maintain surface expression of anti-CD3 after being pre-incubated with anti-CD3 antibody (16). One day after in vitro coincubation, CAR Tregs and CAR Teff cells were enriched using human CD4+ magnetic positive selection kit, and either processed for bulk RNA-seq libraries (FIGS. 4-10, 35) or for 10X Genomics single-cell RNA-seq (FIGS. 11-22, 24-26, 34, 36,37).

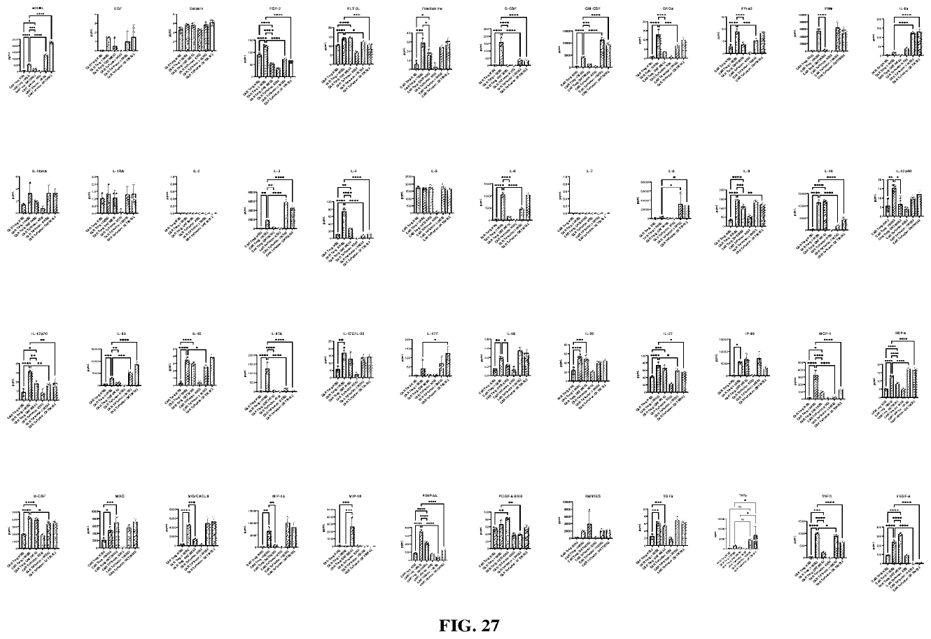

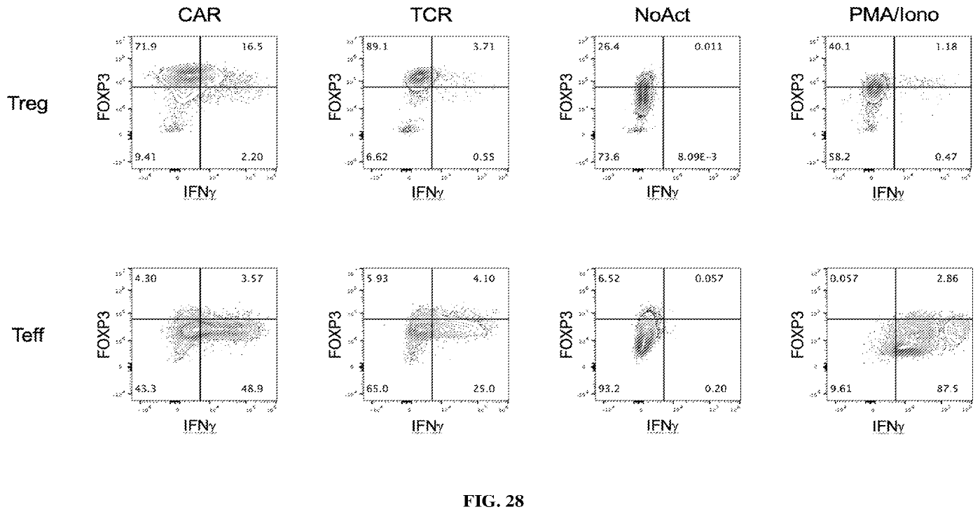

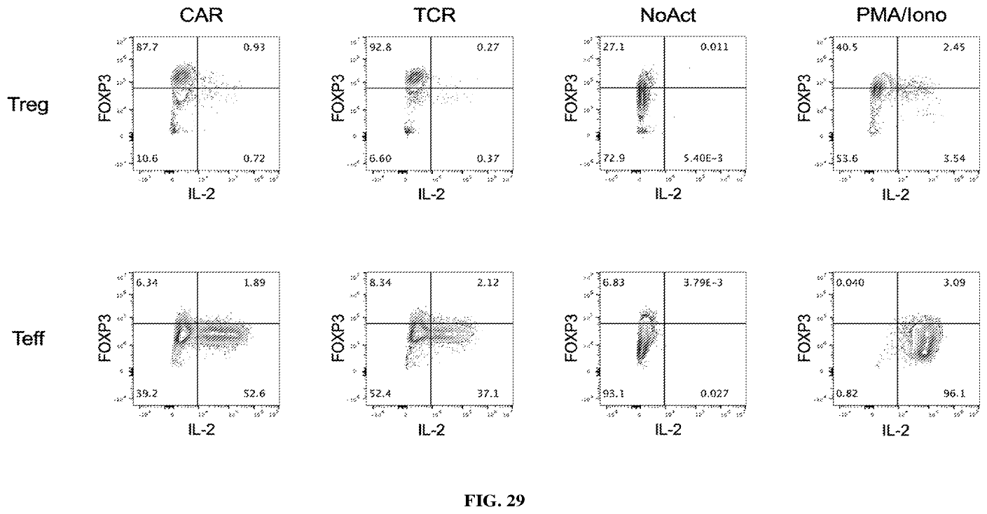

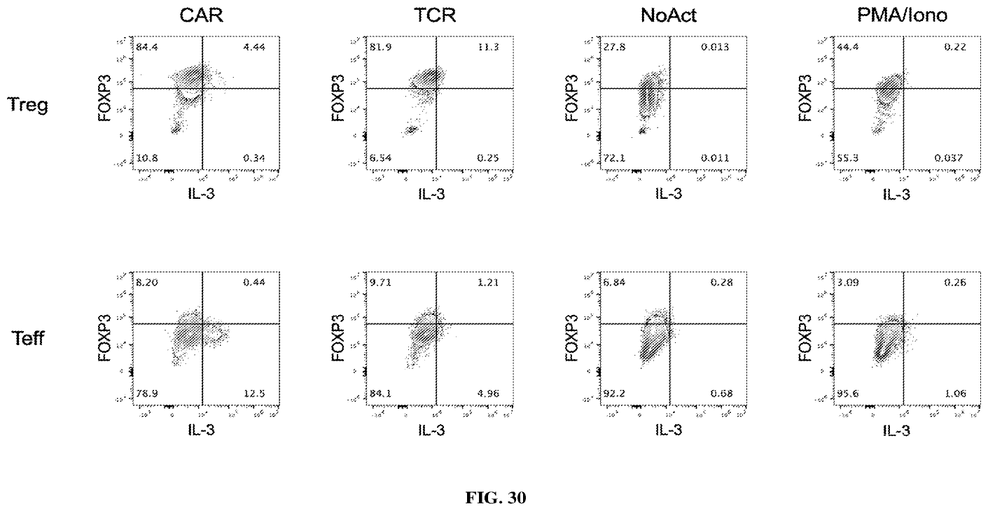

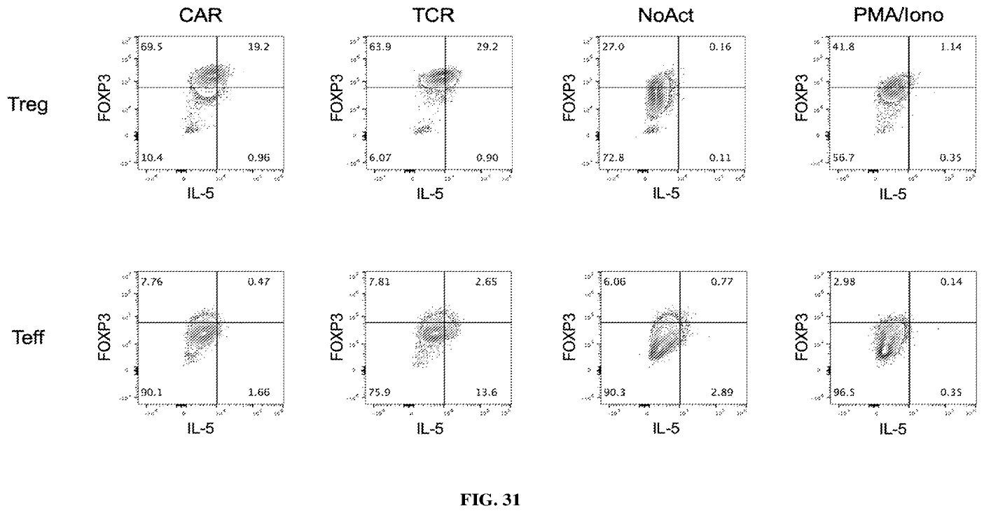

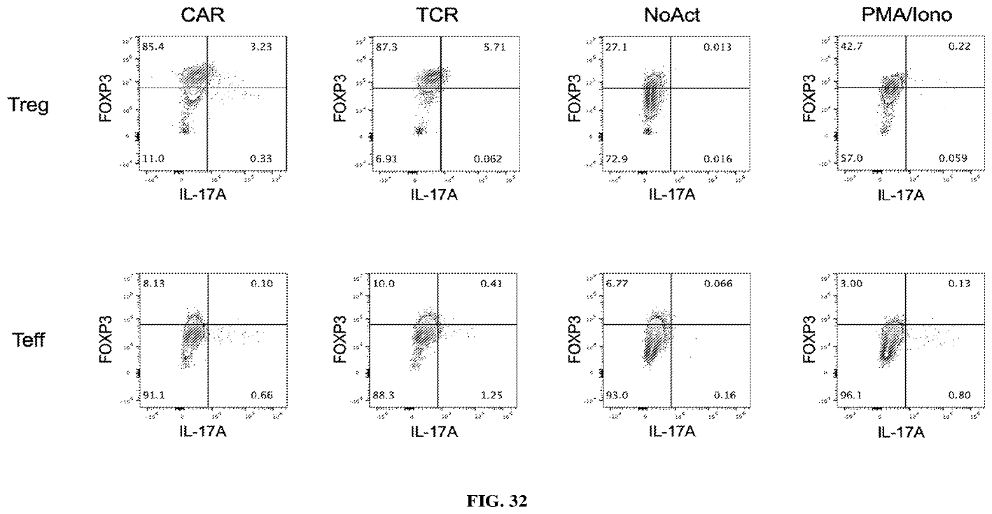

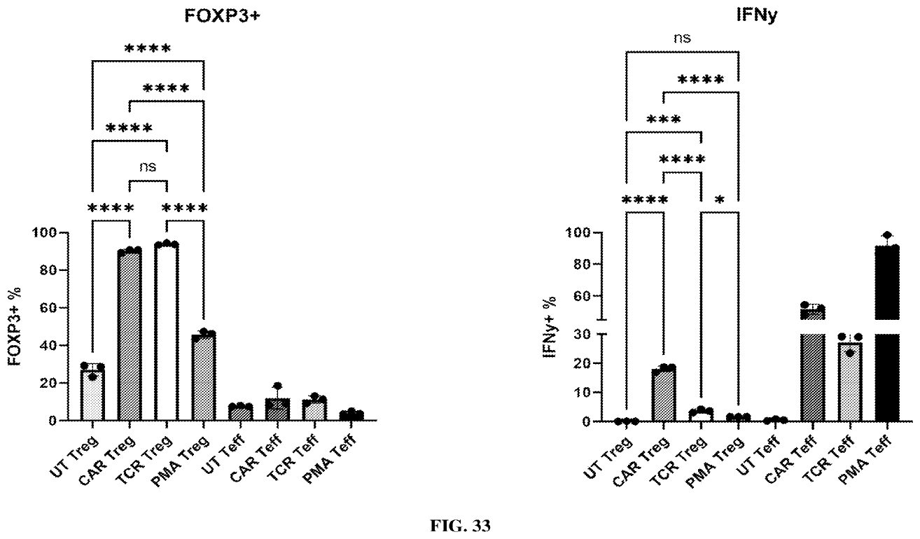

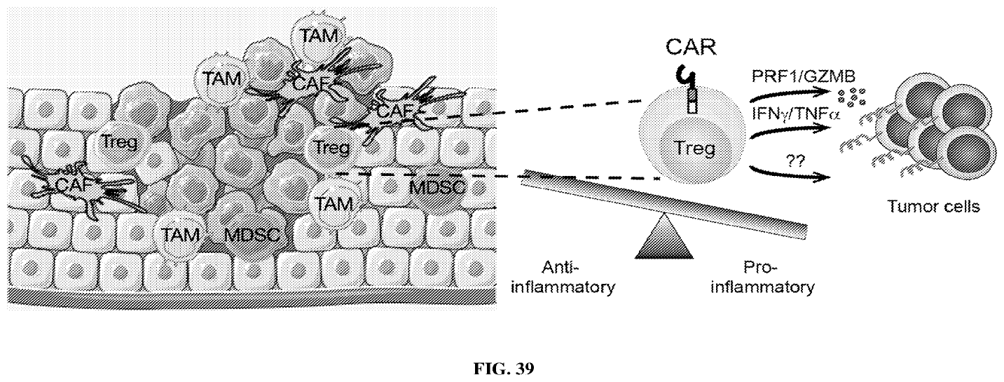

For cytokine secretion analysis (FIG. 27), CAR Tregs and CAR Teff were co-incubated with irradiated target cell lines as described above and supernatants collected 48h later and shipped to EveTech Technologies for 48-plex cytokine quantification. For cytokine production coupled with FOXP3 protein expression analysis at the single-cell level (FIGS. 28-33), CAR Tregs and CAR Teff were co-incubated with irradiated target cell lines as described above overnight, followed by incubation with brefeldin A, intracellular staining, and analysis by flow cytometry. For FIG. 39, C57BL/6 mouse splenocytes were isolated, CD4+CD25+ Tregs and CD4+CD25− Tconv cells magnetically purified from the splenocytes and activated with anti-mouse CD3/CD28 beads and recombinant human IL-2 (2,000 IU/ml IL-2 for Treg and 100 IU/ml IL-2 for Tconv). Two days later, mouse Treg and Tconv were de-beaded and transduced with anti-mouse CD19 mCD28-CD3zeta CAR encoding retrovirus. After one week of in vitro expansion in the presence of recombinant human IL-2, cells were used for assays For FIG. 39A, mouse CD19 CAR Treg or mouse CD19 CAR Tconv or un-tranduced (UT) Tconv cells were co-incubated with A20 mouse lymphoma cells at different ratios and lactate dehydrogenase (LDH) release measured two days later as a measurement of cell death. For FIG. 39B, mouse CD19 CAR Tregs were co-incubated with CELLTRACE™ Violet (CTV) labeled mouse Tconv cells and CTV dilution measured by flow cytometry 3 days later to assess suppression of Tconv cell proliferation. For FIG. 39C, mouse CD19 CAR Tregs were intracellularly stained with anti-mouse Foxp3 APC and analyzed using flow cytometry.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1 Ferreira, L. M. R., Muller, Y. D., Bluestone, J. A., and Tang, Q. Z. 2019. Next-generation regulatory T cell therapy. Nat Rev Drug Discov 18:749.
2 Ohue, Y. and Nishikawa, H. 2019. Regulatory T (Treg) cells in cancer: Can Treg cells be a new therapeutic target? Cancer Sci 110:2080.
3 June, C. H. and Sadelain, M. 2018. Chimeric Antigen Receptor Therapy. N Engl J Med 379:64.
4 Rodriguez-Garcia, A., Palazon, A., Noguera-Ortega, E., Powell Jr., D. J., and Guedan, S. 2020. CAR-T Cells Hit the Tumor Microenvironment: Strategies to Overcome Tumor Escape. Front Immunol 11:1109.
5 Wang, Y., Li, X., Mo, Y., Fan, C., Tang, L., Xiong, F., Guo, C., Xiang, B., Zhou, M., Ma, J., Huang, X. M., Wu, X., Li, Y., Li, G., Zeng, Z., and Xiong, W. 2018. Effects of tumor metabolic microenvironment on regulatory T cells. Molecular Cancer 17:168.
6 Tang, Q., Henriksen, K. J., Bi, M., Finger, E. B., Szot, G., Ye, J., Masteller, E. L., McDevitt, H., Bonyhadi, M., and Bluestone, J. A. 2004. In vitro-expanded antigen-specific regulatory T cells suppress autoimmune diabetes. J Exp Med 199:1455.
7 Waldman, A. D., Fritz, J. M., and Lenardo, M. J. 2020. A guide to cancer immunotherapy: from T cell basic science to clinical practice. Nat Rev Immunol 20:651.
8 Boissonnas, A., Scholer-Dahirel, A., Simon-Blancal, V., Pace, L., Valet, F., Kissenpfennig, A., Sparwasser, T., Malissen, B., Fetler, L., and Amigorena, S. 2010. Foxp3+ T cells induce perforin-dependent dendritic cell death in tumor-draining lymph nodes. Immunity 32:266.
9 Ludwig-Portugall, I., Hamilton-Williams, E. E., Gottschalk, C., and Kurts, C. 2008.Cutting edge: CD25+ regulatory T cells prevent expansion and induce apoptosis of B cells specific for tissue autoantigens. J Immunol 181:4447.
10 Cao, X., Cai, S. F., Fehniger, T. A., Song, J., Collins, L. I., Piwnica-Worms, D. R., and Ley, T. J. 2007. Granzyme B and perforin are important for regulatory T cell-mediated suppression of tumor clearance. Immunity 27:635.
11 Esensten, J. H., Helou, Y. A., Chopra, G., Weiss, A., and Bluestone, J. A. 2016. CD28 Costimulation: From Mechanism to Therapy. Immunity 44:973.
12 Wyss, L., Stadinski, B. D., King, C. G., Schallenberg, S., McCarthy, N. I., Lee, J. Y., Kretschmer, K., Terracciano, L. M., Anderson, G., Surh, C. D., Huseby, E. S., and Palmer, E. 2016. Affinity for self-antigen selects T-reg cells with distinct functional properties. Nat Immunol 17:1093.
13 Yan, D., Farache, J., Mingueneau, M., Mathis, D., and Benoist, C. 2015. Imbalanced signal transduction in regulatory T cells expressing the transcription factor FoxP3. Proc Natl Acad Sci USA 112:14942.
14 Holst, J., Wang, H., Eder, K. D., Workman, C. J., Boyd, K. L., Baquet, Z., Singh, H., Forbes, K., Chruscinski, A., Smeyne, R., van Oers, N. S., Utz, P. J., and Vignali, D. A. 2008. Scalable signaling mediated by T cell antigen receptor-CD3 ITAMs ensures effective negative selection and prevents autoimmunity. Nat Immunol 9:658.
15 Liu, W., Putnam, A. L., Xu-Yu, Z., Szot, G. L., Lee, M. R., Zhu, S., Gottlieb, P. A., Kapranov, P., Gingeras, T. R., Fazekas de St Groth, B., Clayberger, C., Soper, D. M., Ziegler, S. F., and Bluestone, J. A. 2006. CD127 expression inversely correlates with FoxP3 and suppressive function of human CD4+ T reg cells. J Exp Med 203:1701.
16 Suhoski, M. M., Golovina, T. N., Aqui, N. A., Tai, V. C., Varela-Rohena, A., Milone, M. C., Carroll, R. G., Riley, J. L., and June, C. H. 2007. Engineering Artificial Antigen-presenting Cells to Express a Diverse Array of Co-stimulatory Molecules. Mol Ther 15:981.
17 He, X., Smeets, R. L., van Rijssen, E., Boots, A. M. H., Joosten, I., and Koenen, H. J. P. M. 2017. Single CD28 stimulation induces stable and polyclonal expansion of human regulatory T cells. Sci Rep 7:43003.
18 Goldstein, J. S., Chen, T., Brunswick, M., Mostwosky, H., and Kozlowski, S. 1998. Purified MHC Class I and Peptide Complexes Activate Naive CD8+ T Cells Independently of the CD28/B7 and LFA-1/ICAM-1 Costimulatory Interactions. J Immunol 160:3180.
19 Brusko, T. M., Koya, R. C., Zhu, S., Lee, M. R., Putnam, A. L., McClymont, S. A., Nishimura, M. I., Han, S., Chang, L. J., Atkinson, M. A., Ribas, A., and Bluestone, J. A. 2010. Human antigen-specific regulatory T cells generated by T cell receptor gene transfer. PLoS One 5:e11726.
20 Russell, J. H. and Ley, T. J. 2002. Lymphocyte-mediated cytotoxicity. Annu Rev Immunol 20:323.
21 Benmebarek, M. R., Karches, C. H., Cadilha, B. L., Lesch, S., Endres, S., and Kobold, S. 2019. Killing Mechanisms of Chimeric Antigen Receptor (CAR) T Cells. Int J Mol Sci 20.
22 Muller, Y. D., Ferreira, L. M. R., Ronin, E., Ho, P., Nguyen, V., Faleo, G., Zhou, Y., Lee, K., Leung, K. K., Skartsis, N., Kaul, A. M., Mulder, A., Claas, F. H. J., Wells, J. A., Bluestone, J. A., and Tang, Q. 2021. Precision Engineering of an Anti-HLA-A2 Chimeric Antigen Receptor in Regulatory T Cells for Transplant Immune Tolerance. Front Immunol 12:686439.
23 Brinkman, E. K., Chen, T., Amendola, M., and van Steensel, B. 2014. Easy quantitative assessment of genome editing by sequence trace decomposition. Nucleic Acids Res 42: e168.
24 Guipouy, D., Gertner-Dardenne, J., Pfajfer, L., German, Y., Belmonte, N., and Dupre, L. 2019. Granulysin—and granzyme-dependent elimination of myeloid cells by therapeutic ova-specific type 1 regulatory T cells. Int Immunol 31:239.
25 Velaga, S., Ukena, S. N., Dringenberg, U., Alter, C., Pardo, J., Kershaw, O., and Franzke, A. 2015. Granzyme A Is Required for Regulatory T-Cell Mediated Prevention of Gastrointestinal Graft-versus-Host Disease. PLoS One 10: e0124927.
26 Bellin, M. D., Barton, F. B., Heitman, A., Harmon, J. V., Kandaswamy, R., Balamurugan, A. N., Sutherland, D. E., Alejandro, R., and Hering, B. J. 2012. Potent induction immunotherapy promotes long-term insulin independence after islet transplantation in type 1 diabetes. Am J Transplant 12:1576.
27 Choi, B. D., Gedeon, P. C., Herndon, J. E., Archer, G. E., Reap, E. A., Sanchez-Perez, L., Mitchell, D. A., Bigner, D. D., and Sampson, J. H. 2013. Human Regulatory T Cells Kill Tumor Cells through Granzyme-Dependent Cytotoxicity upon Retargeting with a Bispecific Antibody. Cancer Immunology Research 1:163.
28 Levine, A. G., Arvey, A., Jin, W., and Rudensky, A. Y. 2014. Continuous requirement for the TCR in regulatory T cell function. Nat Immunol 15:1070.
29 Stenger, D., Stief, T. A., Kaeuferle, T., Willier, S., Rataj, F., Schober, K., Vick, B., Lotfi, R., Wagner, B., Grunewald, T. G. P., Kobold, S., Busch, D. H., Jeremias, I., Blaeschke, F., and Feuchtinger, T. 2020. Endogenous TCR promotes in vivo persistence of CD19-CAR-T cells compared to a CRISPR/Cas9-mediated TCR knockout CAR. Blood 136:1407.

What is claimed:

1. A method of treating cancer in a subject comprising administering to the subject an effective amount of chimeric antigen receptor (CAR) regulatory T cells (T_{regs}).

2. The method of claim 1, wherein the CAR comprises a CD28-CD3ξ intracellular domain.

3. The method of claim 1 or 2, wherein the CAR binds a tumor-associated antigen.

4. The method of claim 3, wherein the tumor-associated antigen is selected from the group consisting of CD19, CD20, CD22, B-cell maturation antigen (BCMA), carcinoembryonic antigen (CEA), alphafetoprotein, CA-125, MUC-1, epithelial tumor antigen, melanoma-associated antigen (MAGE), mutated p53-derived peptide-HLA, mutated ras-derived peptide-HLA, HER2/Neu, ERBB2, folate binding protein, HIV-1 envelope glycoprotein gp120, HIV-1 envelope glycoprotein gp41, GD2, CD123 (IL3RA), CD319, CD23, CD30, CD56, c-Met, mesothelin, GD3, HERV-K, IL-11Ralpha, kappa chain, lambda chain, CSPG4, ERBB2, EGFR, EGFRvIII, VEGFR2, TNFRSF17, SDC1, FAP, CD44, MS4A1,EPCAM, CA9, CD174, TNFRSF8, CD33, CD38, EPHA2, CD248, CD274, CD276, CD5, NCAM1, CD70, ERBB2, KDR, L1CAM, ULBP1, ULBP2, IL1RAP, GPC3, IL13RA2, ROR1, CEACAM5, MET, FOLH1, CSPG4, CD133, GPNMB, and PSCA.

5. The method of any of claims 1-4, wherein the T_{regs} are human T_{regs}.

6. The method of claim 5, wherein the human T_{regs} were isolated from human peripheral blood by sorting for CD4⁺CD25^{high}CD127^{low} cells.

7. The method of any of claims 1-6, wherein the cancer is acute lymphoblastic leukemia (ALL), B cell leukemia, myeloid leukemia, or epithelial lung carcinoma.

8. The method of any of claims 1-6, wherein the cancer is oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer.

9. The method of any of claims 1-8, wherein the T cells and/or at least one additional therapeutic agent is administered intravenously, intraperitoneally, intratracheally, intratumorally, intramuscularly, endoscopically, intralesionally, percutaneously, subcutaneously, regionally, or by direct injection or perfusion.

10. The method of any of claims 1-9, wherein the CAR T_{regs} are delivered intravenously or subcutaneously.

11. The method of any of claims 1-10, wherein the CAR T_{regs} express IFN-γ, TNF-α, perforin and/or granzyme B.

12. The method of any of claims 1-11, wherein the CAR T_{regs} express pro-inflammatory cytokines IFN-γ, IL-3, CXCL9, CXCL11, IL-2, IL-9, IL-17A, CSF3, CCL3, TNFα, and/or IL-6.

13. The method of any of claims 1-12, wherein the CAR T_{regs} express cytolytic proteins granzyme A, granzyme B, perforin 1 (PRF1), NKG7, and/or granzyme H.

14. The method of any of claims 1-13, wherein the CAR T_{regs} secrete IL-10.

15. The method of any of claims 1-14, wherein the CAR T_{regs} express FOXP3, CD25, BATF, ICOS, GITR, and/or a demethylated T_{reg}-specific demethylated region (TSDR).

16. The method of any of claims 1-15, wherein the CAR T_{regs} are conjugated to a cytotoxic agent.

17. The method of claim 16, wherein the cytotoxic agent is a chemotherapeutic, IL-2, IL-15, soluble TRAIL, perforin, or granzyme B.

18. The method of any of claims 1-17, further comprising administering at least a second anticancer therapy to the subject.

19. The method of claim 18, wherein the second anticancer therapy is a surgical therapy, chemotherapy, radiation therapy, cryotherapy, hormonal therapy, immunotherapy or cytokine therapy.

20. The method of any of claims 1-19, wherein the CAR T_{regs} are administered more than once.

21. A composition comprising T_{regs} engineered to express a CAR construct.

22. The composition of claim 21, wherein the CAR construct comprises a tumor-associated antigen antibody or fragment thereof selected from the group consisting of F(ab′)2, Fab′, Fab, Fv, and scFv.

23. The composition of claim 21, wherein the CAR binds a tumor-associated antigen.

24. The composition of claim 23, wherein the tumor-associated antigen is selected from the group consisting of CD19, CD20, CD22, B-cell maturation antigen (BCMA), carcinoembryonic antigen (CEA), alphafetoprotein, CA-125, MUC-1, epithelial tumor antigen, melanoma-associated antigen (MAGE), mutated p53-derived peptide-HLA, mutated ras-derived peptide-HLA, HER2/Neu, ERBB2, folate binding protein, HIV-1 envelope glycoprotein gp120, HIV-1 envelope glycoprotein gp41, GD2, CD123 (IL3RA), CD319, CD23, CD30, CD56, c-Met, mesothelin, GD3, HERV-K, IL-11Ralpha, kappa chain, lambda chain, CSPG4, ERBB2, EGFR, EGFRvIII, VEGFR2, TNFRSF17, SDC1, FAP, CD44, MS4A1, EPCAM, CA9, CD174, TNFRSF8, CD33, CD38, EPHA2, CD248, CD274, CD276, CD5, NCAM1, CD70, ERBB2, KDR, LICAM, ULBP1, ULBP2, IL1RAP, GPC3, IL13RA2, RORI, CEACAM5, MET, FOLH1, CSPG4, CD133, GPNMB, and PSCA.

25. The composition of claim 21, wherein the T_{regs} are autologous.

26. The composition of claim 21, wherein the T_{regs} are allogeneic.

27. The composition of any of claims 21-26, wherein the T_{regs} express pro-inflammatory cytokines IFN-γ, IL-3, CXCL9, CXCL11, IL-2, IL-9, IL-17A, CSF3, CCL3, TNFα, and/or IL-6.

28. The composition of any of claims 21-27, wherein the T_{regs} express cytolytic proteins granzyme A, granzyme B, perforin 1 (PRF1), NKG7, and/or granzyme H.

29. The composition of any of claims 21-28, wherein the T_{regs} secrete IL-10.

30. The composition of any of claims 21-29, wherein the T_{regs} express FOXP3, CD25, BATF, ICOS, GITR, and/or a demethylated T_{reg}-specific demethylated region (TSDR).

31. The composition of any of claims 21-30, wherein the composition is essentially free of CD8⁺ T cells.

32. The composition of any of claims 21-31, wherein the T_{regs} are conjugated to a cytotoxic agent.

33. The composition of claim 32, wherein the cytotoxic agent is a chemotherapeutic, IL-2, IL-15, soluble TRAIL, perforin, or granzyme B.

34. A pharmaceutical composition comprising the T_{regs} of any of claims 21-33 and a pharmaceutical carrier.

35. A composition comprising an effective amount of T_{regs} of any of claims 21-33 for use in the treatment of cancer in a subject.

36. An in vitro method of generating CAR T_{regs} comprising:
(a) isolating T_{regs} from peripheral blood;
(b) introducing a CAR expression construct to the T_{regs};
(c) expanding the T_{regs} in the presence of at least one cytokine; and
(d) stimulating the T_{regs} with artificial presenting cells (APCs).

37. The method of claim 36, wherein the CAR expression construct is a lentiviral vector or retroviral vector.

38. The method of claim 36, wherein introducing comprises contacting the T_{regs} with lentiviral particles comprising a CAR construct.

39. The method of any of claims 36-38, wherein the at least one cytokine is IL-2.

40. The method of any of claims 36-39, wherein the APCs are gamma-irradiated APCs.

41. The method of any of claims 36-40, wherein the APCs are CD19-K562 cells.

42. The method of any of claims 36-41, wherein the CAR expression construct is a CD19-specific construct.

43. The method of any of claims 36-41, wherein the CAR expression construct is selected from the group consisting of CD19, CD20, CD22, B-cell maturation antigen (BCMA), carcinoembryonic antigen (CEA), alphafetoprotein, CA-125, MUC-1, epithelial tumor antigen, melanoma-associated antigen (MAGE), mutated p53-derived peptide-HLA, mutated ras-derived peptide-HLA, HER2/Neu, ERBB2, folate binding protein, HIV-1 envelope glycoprotein gp120, HIV-1 envelope glycoprotein gp41, GD2, CD123 (IL3RA), CD319, CD23, CD30, CD56, c-Met, mesothelin, GD3, HERV-K, IL-11Ralpha, kappa chain, lambda chain, CSPG4, ERBB2, EGFR, EGFRvIII, VEGFR2, TNFRSF17, SDC1,FAP, CD44, MS4A1, EPCAM, CA9, CD174, TNFRSF8, CD33, CD38, EPHA2, CD248, CD274, CD276, CD5, NCAMI, CD70, ERBB2, KDR, LICAM, ULBP1, ULBP2, IL1RAP, GPC3, IL13RA2, ROR1, CEACAM5, MET, FOLH1, CSPG4, CD133, GPNMB, and PSCA CAR expression construct.
